(19) United States
(12) Patent Application Publication
(10) Pub. No.: US 2005/0119167 A1
(43) Pub. Date: Jun. 2, 2005

(54) Process for the preparation of cyclic peptides

(76) Inventors: Giovanni Abbenante, Samsonvale (AU); David P. Fairlie, (US); Robert C. Reid, Chelmer (AU); Hua Ming Williams, Brisbane (AU)

Correspondence Address: WILLIAMS, MORGAN & AMERSON, P.C., 10333 RICHMOND, SUITE 1100, HOUSTON, TX, 77042, US

(21) Appl. No.: 10/839,760

(22) Filed: May 5, 2004

(30) Foreign Application Priority Data
Jun. 2, 2003 (AU) 2003902743

(51) Int. Cl. A61K 38/12; C07K 5/12

(52) U.S. Cl. 514/9; 530/317

(57) ABSTRACT

The present invention relates to a process for the preparation of cyclic peptides, in particular the preparation of Ac-Phe[Orn-Pro-D-Cha-Trp-Arg] known as 3D53 or PMX53 which is a macrocyclic peptidomimetic of the human plasma protein C5a and displays excellent anti-inflammatory activity.

The present application claims priority to co-pending Australian application number 2003902743, filed Jun. 2, 2003, the entire disclosure of which application is specifically incorporated herein by reference without disclaimer.

### FIELD

The present invention relates to a process for the preparation of cyclic peptides, in particular the preparation of Ac-Phe[Orn-Pro-D-Cha-Trp-Arg] known as 3D53 or PMX53 which is a macrocyclic peptidomimetic of the human plasma protein C5a and displays excellent anti-inflammatory activity.

### BACKGROUND

An important part of the human immune system is a set of blood proteins termed Complement. One of these proteins, known as C5a, regulates many types of human immune and other cells by binding to a specific receptor on the cell surface, triggering cellular immune responses and release of numerous inflammatory mediators¹. However, overexpression or underregulation of C5a is implicated in the pathogenesis of many immunoinflammatory conditions, such as, rheumatoid- and osteo-arthritis, Alzheimer's disease, cystic fibrosis, tissue graft rejection, ischaemic heart disease, psoriasis, gingivitis, atherosclerosis, lung injury, fibrosis, systemic lupus erythematosus, reperfusion injury, and major systemic disturbances such as septic and anaphylactic shock, burns, and major trauma or infection that leads to adult respiratory distress syndrome². Medical conditions that arise from excessive complement activation are known to affect hundreds of millions of people and represent annual multibillion dollar pharmaceutical market opportunities in the USA alone³.

The importance of a turn conformation in the recognition of the C-terminus of C5a by its G protein-coupled receptor was recently found. From the structure⁴ of a truncated hexapeptide derivative, a cyclic antagonist Ac-Phe[Orn-Pro-D-Cha-Trp-Arg] known as 3D53 or PMX53 which is a macrocyclic peptidomimetic of the human plasma protein C5a 1 was prepared to stabilise the putative turn structure which was believed to be involved in receptor binding⁵. Compound 1, featuring an i→i+4 side chain (ornithine-δNH₂) to main chain (arginine-CO₂H) amide bond linkage, was originally created as a molecular probe to identify key features needed for construction of a non-peptidic drug candidate. This macrocyclic compound proved to be the first potent, selective, and orally active antagonist of the human C5a receptor with potent inhibition in vitro⁶⁻⁹ of C5a binding to human cells, and C5a-mediated activation of neutrophils and macrophages, chemotaxis, and cytokine release from polymorphonuclear leukocytes. Since it also showed potent inhibition in vivo in many rat models of human disease, including neutropenia/sepsis¹⁰, arthritis¹¹, immune-complex dermal inflammation¹², arthus and endotoxic shock¹³, and ischemia-reperfusion injury^{14,15}, it was decided to more extensively evaluate this compound for efficacy in vivo. It was anticipated that much larger quantities (50-100 g) of 1 would be required than could be obtained inexpensively and rapidly by solid-phase approaches.

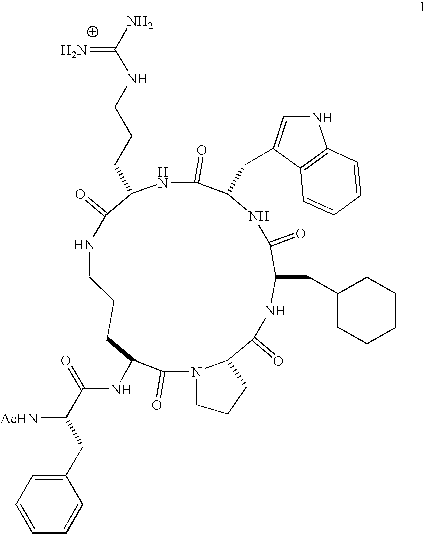

A number of other cyclic peptides have entered the marketplace as drugs, including cyclosporin (immunosuppressant)¹⁶, caspofungan (fungicidal)¹⁷, eptifibatide (antithrombotic)¹⁸, dalfopristin and quinupristine (antibacterials)¹⁸, atosiban (tocolytic)¹⁹, lepirudin (anticoagulant)²⁰, lanreotide (acromegaly)²¹ and octreotide (acromegaly)²¹. Although most cyclic peptides synthesised for research purposes are made on a small scale using conventional Merrifield-based solid phase peptide synthesis methods, larger quantities needed for preclinical and clinical investigations need to be obtained more cheaply. Usually to date this has been through fermentation, but sometimes via solution phase syntheses. Relatively few large-scale solution syntheses of cyclic peptides have been previously reported in the literature^{22,23}, most using a mixed-anhydride method that appears optimal for large-scale peptide couplings in solution. The procedure is efficient, inexpensive and gives high yields with low racemisation at each step. The one report²³, that has dealt with an arginine-containing peptide, used the tosyl protecting group for the arginine side-chain. This required the use of trifluoromethanesulfonic acid, a very corrosive agent, in the final deprotection step.

The original synthesis^{4,5} of 1 involved a conventional assembly of the linear hexapeptide in small quantities by solid phase peptide synthesis using Fmoc protocols on Arg(Pmc)-Wang resin^{24,25}, followed by cyclization in solution using benzotriazol-1-yloxy-tri(dimethylamino)-phosphonium hexafluorophsophate (BOP). To scale up the synthesis via solution phase, a plan to realize high yields from inexpensive reagents, to minimize purification steps, and to avoid racemization was needed.

### SUMMARY

It was decided that the synthesis of 1 would be most efficient via a convergent approach, involving synthesis and coupling of the component tripeptides Ac-Phe-Orn(Boc)-Pro-OH 2 and H-D-Cha-Trp(For)-Arg-OEt 3 to give the linear hexapeptide, which could then be cyclised. 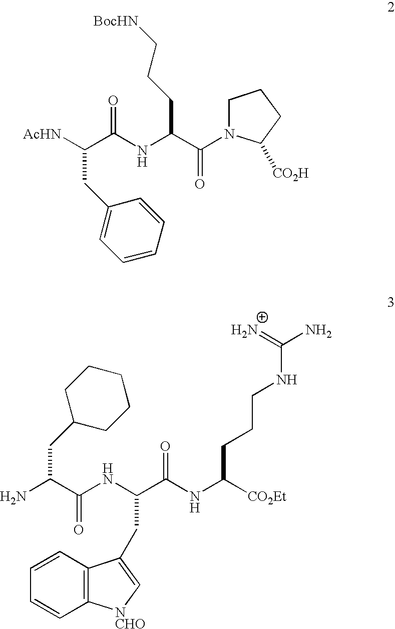
The solution phase synthesis of 1 uses cheap reagents, requires no purification of intermediates and delivers reasonable yields of the required product in 50-100 g quantities and in high purity. This process is suitable for the synthesis of 1 and derivatives in a medium to large scale.

According to the present invention there is provided a process for the preparation of a compound of formula I 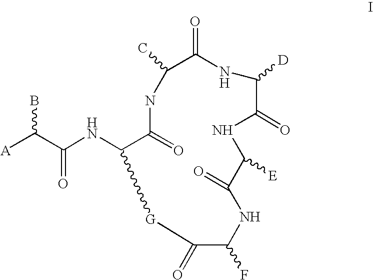
in which
A is H, NH₂, optionally substituted alkyl, optionally substituted aryl, NH acyl, NH optionally substituted alkyl, N(optionally substituted alkyl)₂ or NH succinate;
B is optionally substituted alkyl or optionally substituted aryl;
C is an optionally protected amino acid side chain;
D is an optionally protected amino acid side chain;
E is an optionally protected amino acid side chain; optionally substituted aryl; or optionally substituted heteroaryl;
F is an optionally protected D- or L-amino acid side chain selected from the group consisting of arginine, homoarginine, citrulline, homocitrulline, glutamine, lysine and canavanine; and
G is an optionally protected D- or L-amino acid side chain selected from the group consisting of ornithine and lysine,
or pharmaceutically acceptable salts, derivatives, hydrates, solvates, prodrugs, tautomers and/or isomers thereof
which comprises the steps of:
(a) coupling an optionally protected compound of formula II 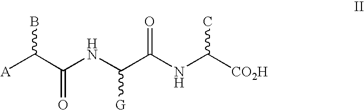
   in which A, B, C and G are as defined in formula I with an optionally protected compound of formula III 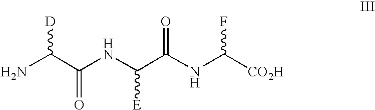
   in which D, E and F are as defined in formula I to form an optionally protected compound of formula IV 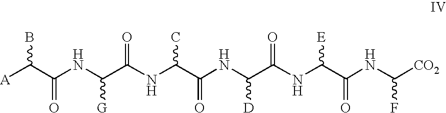
   in which A, B, C, D, E, F and G are as defined in formula I; and
(b) cyclising the compound of formula IV.

The present invention also provides a compound of formula I whenever prepared by the process defined above.

Preferably A is NH acyl or NH succinate.

The optionally substituted aryl in B is preferably an optionally substituted phenyl or an optionally substituted benzyl, more preferably phenyl, benzyl, 4-nitrophenyl, 4-aminophenyl, 4-dimethylaminophenyl, halophenyl or phenyl-(CH₂) n in which n is an integer from 2 to 5.

C is preferably an optionally protected side chain of L- or D-amino proline or hydroxyproline.

Preferably, D is an optionally protected side chain of L- or D-cyclohexane amino acid.

E is preferably an optionally protected side chain of L- or D-tryptophan or alanine. The optionally substituted aryl in E is preferably an optionally substituted naphthyl or an optionally substituted benzothienyl.

In a particularly preferred embodiment, the compound of formula I is Ac-Phe[Orn-Pro-D-Cha-Trp-Arg] 1 known as 3D53.

The compounds of formulas II, III and IV used to prepare the particularly preferred compound 1 are Ac-Phe-Orn(Boc)-Pro-OH 2, D-Cha-Trp(For)-Arg-OEt 3 and compounds 22-24 shown below, respectively.

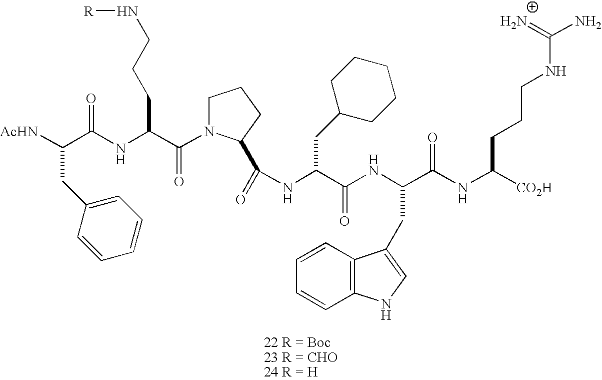

The intermediate compounds of formulae II and III as defined above are also novel and form part of the present invention.

The present invention further provides a process for the preparation of the compound of formula II defined above which comprises coupling an optionally protected compound of formula V 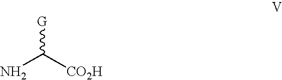
in which G is as defined in formula I
and an optionally protected compound of formula VI 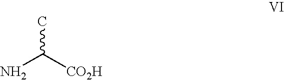
in which C is as defined in formula I above
and an optionally protected compound of formula VII 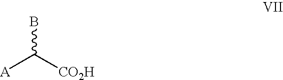
in which A and B are as defined in formula I above.

Preferably the compound of formula V is first coupled with the compound of formula VI to form a dipeptide which is then coupled to the compound of formula VII.

The present invention still further provides a process for the preparation of the compound of formula III as defined above which comprises coupling an optionally protected compound of formula VIII 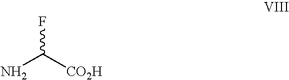
in which F is as defined in formula I
and an optionally protected compound of formula IX 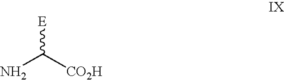
in which E is as defined in formula I
and an optionally protected compound of formula X 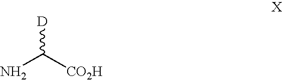
in which D is as defined in formula I.

The compound of formula VIII is preferably first coupled with the compound of formula IX to form a dipeptide which is then coupled to the compound of formula X.

In a particularly preferred embodiment, the compounds of the formulae II and III are Ac-Phe-Orn(Boc)—Pro-OH 2 and D-Cha-Trp(For)Arg-OEt 3. The formulae V, VI, VII, VIII, IX and X used to prepare the particularly preferred compounds 2 and 3 are Boc-Orn(Cbz)-OH 4, H-Pro-OMe 5, Boc-Phe-OH 13, H-Arg-OEt.2HCl 17, Trp(For)-OH 16 and Boc-D-cyclohexylalanine 15, respectively.

### DETAILED DESCRIPTION

For the purposes of this specification it will be clearly understood that the word “comprising” means “including but not limited to”, and that the word “comprises” has a corresponding meaning.

It must be noted that, as used in the subject specification, the singular forms “a”, “an” and “the” include plural aspects unless the context clearly dictates otherwise. Thus, for example, reference to “a compound of formula I, II, III or IV” includes a single compound, as well as two or more compounds; and so forth.

The term “alkyl” embraces linear, branched or cyclic radicals having 1 to about 20 carbon atoms, preferably, 1 to about 12 carbon atoms. More preferred alkyl radicals have 1 to about 10 carbon atoms and cycloalkyl radicals have 3 to about 8 carbon atoms. Most preferred are alkyl radicals having 1 to about 6 carbon atoms. Examples of such radicals include methyl, ethyl, n-propyl, isopropyl, n-butyl, isobutyl, sec-butyl, tert-butyl, pentyl, iso-amyl, hexyl and the like. Examples of cycloalkyl radicals include cyclopropyl, cyclobutyl, cyclopentyl and cyclohexyl.

The term “aryl” means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pvolyl, hexanoyl and trifluoroacetyl.

The term “heteroaryl” refers to a 5- or 6-membered substituted or unsubstituted aromatic heterocycle containing one or more heteroatoms selected from N, O and S. Illustrative of such rings are thienyl, furyl, imidazolyl, oxadizolyl, pyridyl or pyrazinyl.

The term “halo” refers to fluorine, chlorine, bromine or iodine.

The term “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, alkynyloxy, aryloxy, carboxy, benzyloxy, haloalkoxy, haloalkenyloxy, haloalkynyloxy, haloaryloxy, nitro, nitroalkyl, nitroalkenyl, nitroalkynyl, nitroaryl, nitroheterocyclyl, azido, amino, alkylamino, alkenylamino, alkynylamino, arylamino, benzylamino, acyl, alkenyacyl, alkynylacyl, arylacyl, acylamino, acyloxy, aldehydo, alkylsulphonyl, arylsulphonyl, alkysulphonylamino, arylsulphonylamino, alkylsulphonyloxy, arylsulphonyloxy, heterocyclyl, heterocycloxy, heterocyclylamino, haloheterocyclyl, alkylsulphenyl, arylsulphenyl, carboalkoxy, carboaryloxy, mercapto, alkylthio, arylthio, acylthio and the like.

The term “amino acid side chain” is used in its broadest sense and refers to the side chains of both L- and D-amino acids including the 20 common amino acids such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine; and the less common amino acids but known derivatives such as homo-amino acids, N-alkyl amino acids, dehydro amino acids, aromatic amino acids and α,α-disubstituted amino acids, for example, cystine, 5 hydroxylysine, 4-hydroxyproline, α-aminoadipic acid, α-amino-n-butyric acid, 3,4-dihydroxyphenylalanine, homoserine, α-methylserine, ornithine, pipecolic acid, ortho, meta or para-aminobenzoic acid, citrulline, canavanine, norleucine, δ-glutamic acid, aminobutyric acid, L-fluorenylalanine, L-3-benzothienylalanine and thyroxine; and any amino acid having a molecular weight less than about 500.

The term “optionally protected” is used herein in its broadest sense and refers to an introduced functionality which renders a particular functional group, such as a hydroxy, amino, carbonyl or carboxy group, unreactive under selected conditions and which may later be optionally removed to unmask the functional group. A protected amino acid side chain is one in which the reactive substituents of the side chain or the amino group or carbonyl group of the amino acid are protected. Suitable protecting groups are known in the art and include those disclosed in Greene, T. W., “Protective Groups in Organic Synthesis” John Wiley & Sons, New York 1999, (the contents of which are incorporated herein by reference) as are methods for their installation and removal.

Preferably the N-protecting group is a carbamate such as, 9-fluorenylmethyl carbamate (Fmoc), 2,2,2-trichloroethyl carbamate (Troc), t-butyl carbamate (Boc), allyl carbamate (Alloc), 2-trimethylsilylethyl (Teoc) and benzyl carbamate (Cbz), more preferably Boc or Cbz.

The carbonyl protecting group is preferably an ester such as an alkyl ester, for example, methyl ester, ethyl ester or t-Bu ester or a benzyl ester.

The amino acid side chains may be protected, for example, the carboxyl groups of aspartic acid, glutamic acid and α-aminoadipic acid may be esterified (for example as a C₁-C₆ alkyl ester), the amino groups of lysine, ornithine and 5-hydroxylysine, may be converted to carbamates (for example as a C(═O)OC₁-C₆ alkyl or C(═O)OCH₂Ph carbamate) or imides such as thalimide or succinimide, the hydroxyl groups of 5-hydroxylysine, 4-hydroxyproline, serine, threonine, tyrosine, 3,4-dihydroxyphenylalanine, homoserine, α-methylserine and thyroxine may be converted to ethers (for example a C₁-C₆ alkyl or a (C₁-C₆ alkyl)Phenyl ether) or esters (for example a C═OC₁-C₆ alkyl ester) and the thiol group of cysteine may be converted to thioethers (for example a C₁-C₆ alkyl thioether) or thioesters (for example a C(═O)C₁-C₆ alkyl thioester).

The salts of the compound of formula I are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

In addition, some of the compounds of the present invention may form solvates with water or common organic solvents. Such solvates are encompassed within the scope of the invention.

By “derivative” is meant any salt, hydrate, protected form, ester, amide, active metabolite, analogue, residue or any other compound which is not biologically or otherwise undesirable and induces the desired pharmacological and/or physiological effect. Preferably the derivative is pharmaceutically acceptable.

The term “tautomer” is used in its broadest sense to include compounds of formula I which are capable of existing in a state of equilibrium between two isomeric forms. Such compounds may differ in the bond connecting two atoms or groups and the position of these atoms or groups in the compound.

The term “isomer” is used in its broadest sense and includes structural, geometric and stereo isomers. As the compound of formula I may have one or more chiral centres, it is capable of existing in enantiomeric forms.

The coupling step (a) may be performed using any suitable known technique, preferably involving a coupling agent and a base such as BOP and diphenylphosphonylazide (DPPA). This step is followed by complete and/or partial deprotection if necessary prior to cyclisation using deprotecting agents, such as, NaOH and HCl/dioxane.

The cyclisation step (b) requires activation and coupling of the peptidyl-F residue and was expected to proceed with some degree of racemisation. Considerable effort was directed towards minimising racemisation because separation of diastereomers of formula I was known to be difficult. While it will be appreciated that any known coupling agent and base could be used for the cyclisation, such as BOP/DPPA, BOP/diisopropylethyleneamine (DIPEA), BOP/NaHCO₃ and BOP/tetramethylethylenediamine (TMEDA), the combination of BOP as the coupling agent with DIPEA as the base was found to be optimal.

The cyclisation step (b) may be carried out in a suitable solvent such as an organic solvent, for example, dimethyl formamide (DMF) and is preferably performed at lower temperatures such as about −10° C. to about room temperature so as to minimise racemisation.

The resultant crude product may be extracted and precipitated preferably using diethyl ether. Purification of the final product can be achieved using any suitable known technique, such as, chromatography, for example, preparative HPLC which may be performed more than once to remove any acetate or TFA salt. Suitable buffers for the preparative HPLC include AcOH in water and AcOH in ACN.

The coupling step to prepare the compounds of formulae II and III may be performed using the mixed anhydride method involving ethyl chloroformate as the coupling agent and NMM(N-methyl morpholine) as the base. Other coupling agents can be used such as HBTU (N,N,N′,N′-tetramethyl-O-(1H-benzotriazol-1-yl) uranium hexafluorophosphate), TOTU (O[ethoxycarbonyl) cyanomethylenamino]N,N,N′,N′-tetramethyl uranium tetrafluoroborate), EDC (N-(3-dimethylaminopropyl)-N′-ethylcarbodiimide hydrochloride) or DCC(N,N′-dicyclohexycarbodiimide). HBTU is preferred as the peptide bond formation is complete in about 1 hour in comparison with about 10 hours or even longer for other reagents, the yield is higher and there is very low racemisation. DIPEA is the preferred base for the coupling step.

### DESCRIPTION OF DRAWINGS

In the examples, reference will be made to the accompanying drawings, in which:

FIG. 1 is an ¹⁹F NMR of the crude product having two TFA salts;

FIG. 2 is an ¹H NMR of the crude product without TFA salts; and

FIG. 3 is an ¹⁹F NMR of the crude product without TFA salts.

### EXAMPLES

The invention will now be described with reference to the following non-limiting examples.

### Example 1

### Synthesis of Tripeptide Fragments 2 and 3

Tripeptide Ac-Phe-Orn(Boc)-Pro-OH 2 was prepared as shown in Scheme 1. Boc-Orn(Cbz)-OH 4 was first coupled to H-Pro-OMe 5. Following removal of the Boc protecting group with TFA, the product 7 was then coupled to Boc-Phe-OH 13 to give the tripeptide unit 8. After subsequent removal of the Boc group, the N-terminus was acetylated with Ac₂O to give 10. It was necessary to replace the ornithine side chain Cbz group with Boc for compatibility with subsequent steps. This was accomplished by hydrogenation to give the free amine 11 that was conveniently separated from neutral impurities by extraction into an aqueous phase as the hydrochloride salt and washing with ether. After basification, the amine 11 was treated with Boc₂O to give 12. The C-terminal methyl ester of 12 was hydrolysed with NaOH and, after acidification and extraction, the required tripeptide 2 was obtained as a colourless brittle glass (90% overall yield) that was easily ground to a powder that stored well.

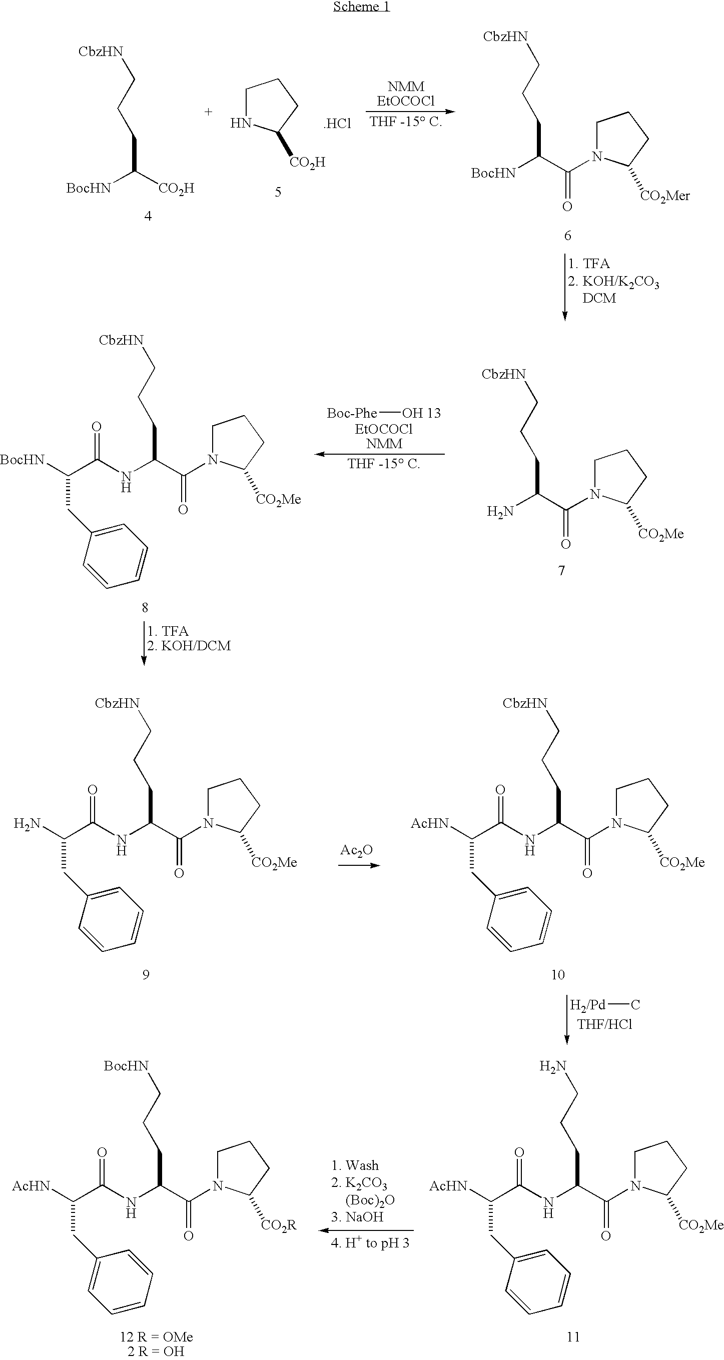

The second tripeptide H-D-Cha-Trp(For)-Arg-OEt.HSO₄ 3 presented several challenges due to side chain functionality. The C-terminal arginine residue traditionally requires protection of the side chain guanidine group, as a sulfonamide (e.g. tosyl) or perhaps by nitration, to prevent unwanted acylation during the several subsequent coupling reactions resulting in a complex mixture. There was a concern however that such protecting groups may prove difficult to remove from the final product under mild conditions. For example, the use of trifluoromethane sulfonic acid or HF were not considered practical due to the difficulty of handling for removal of the tosyl group and the often protracted hydrogenations needed to cleave nitroarginine might not proceed to completion or may effect reduction of the tryptophan indole nucleus. For these reasons conditions were developed that would allow side chain un-protected H-Arg-OEt.2HCl 17 to be employed. It was reasoned that the much greater basicity of the guanidine group (pKa 13) verses amino groups (pKa 9) should permit the desired regioselective couplings. Boc-Trp(For)-OH 16 was considered to be a suitably protected tryptophan derivative for the initial coupling reaction. The coupling of 16 and 17 was best achieved via the mixed anhydride method with ethyl chloroformate using DMF as solvent. The reaction works well if the ethyl ester of arginine is totally dissolved in the DMF solvent before addition to the mixed anhydride of 16, otherwise substantial acylation does occur at the guanidino side-chain. Pouring the reaction mixture into a solution of 10% KHSO₄ causes precipitation of the dipeptide 18 as a gel which can be filtered, washed and air-dried. To quicken the drying process the wet gel can also be azeotroped with 1-butanol on a rotary evaporator to remove water, and then precipitated from the oil formed by the addition of ether and then air-dried. The reaction was performed several times on 25-50 g batches of Boc-Trp(For)-OH with yields typically in the 70-80% range and of >93% purity.

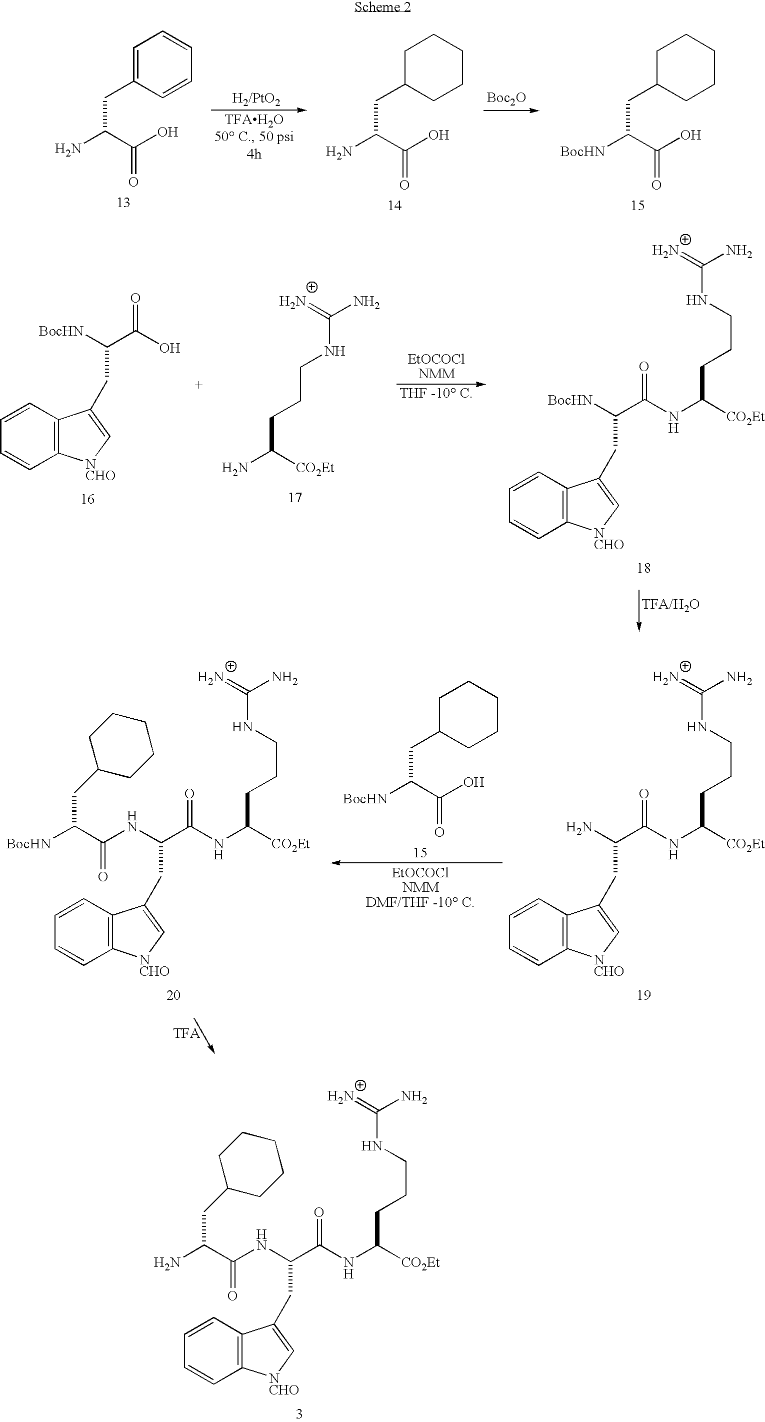

D-Cyclohexylalanine 14 was a reasonably expensive amino acid if sourced commercially and it was sought to develop improved conditions for its preparation from D-phenylalanine 13 by hydrogenation. The use of a solvent consisting of TFA:water 1:1 was found to be the single key factor in improving the efficiency of the PtO₂ catalysed hydrogenation of the aromatic ring over reported literature procedures²⁷. The improvement was largely due to the much greater solubility of the amino acids (13 and 14) in this solvent mixture which enabled concentrations to be kept much higher and avoided poisoning of the catalyst due to precipitation. There is however literature precedence to suggest that TFA increases the overall rate of hydrogenation as compared to HCl, sulfuric acid or acetic acid²⁸. The same charge of catalyst was used for 5×20 g batches of 13 giving complete conversion to 14 within 4 hours with minimal loss of catalytic activity before recycling. D-Cyclohexylalanine 14 was converted to the Boc derivative 15 by standard procedures²⁹ before coupling to H-Trp(For)-Arg-OEt 19 (Scheme 2). The coupling of dipeptide 18 and Boc-D-cyclohexylalanine 14 was achieved by the mixed anhydride method. The tripeptide product 20 could again be purified by simply pouring the reaction mixture into a solution of 10% KHSO₄, filtering, washing and either air-drying or azeotroping with butanol using the same conditions as described for the dipeptide 18. This procedure gave tripeptide 20 with typical yields of 80% and >90% purity.

### Example 2

### Assembly of Linear Hexapeptide from Fragments 2 and 3

Coupling of tripeptides 2 and 3 (Scheme 3) was found to be inefficient using the mixed anhydride method. There remained approximately 20% of unchanged tripeptide 2 together with the ethyl carbamate of tripeptide 3 which was difficult to separate from the desired product 21. An attempt to use the more hindered analogue, isobutyl chloroformate, did not improve the conversion significantly. Ultimately the coupling was achieved cleanly using BOP. The fully protected hexapeptide 21 was found to be very hygroscopic and difficult to handle, and thus was never isolated but deprotected to product 22 which dries to a non-hygroscopic powder after ether precipitation. The coupling and partial deprotection with NaOH was repeated several times giving product 22 in 80% yield and of >94% purity.

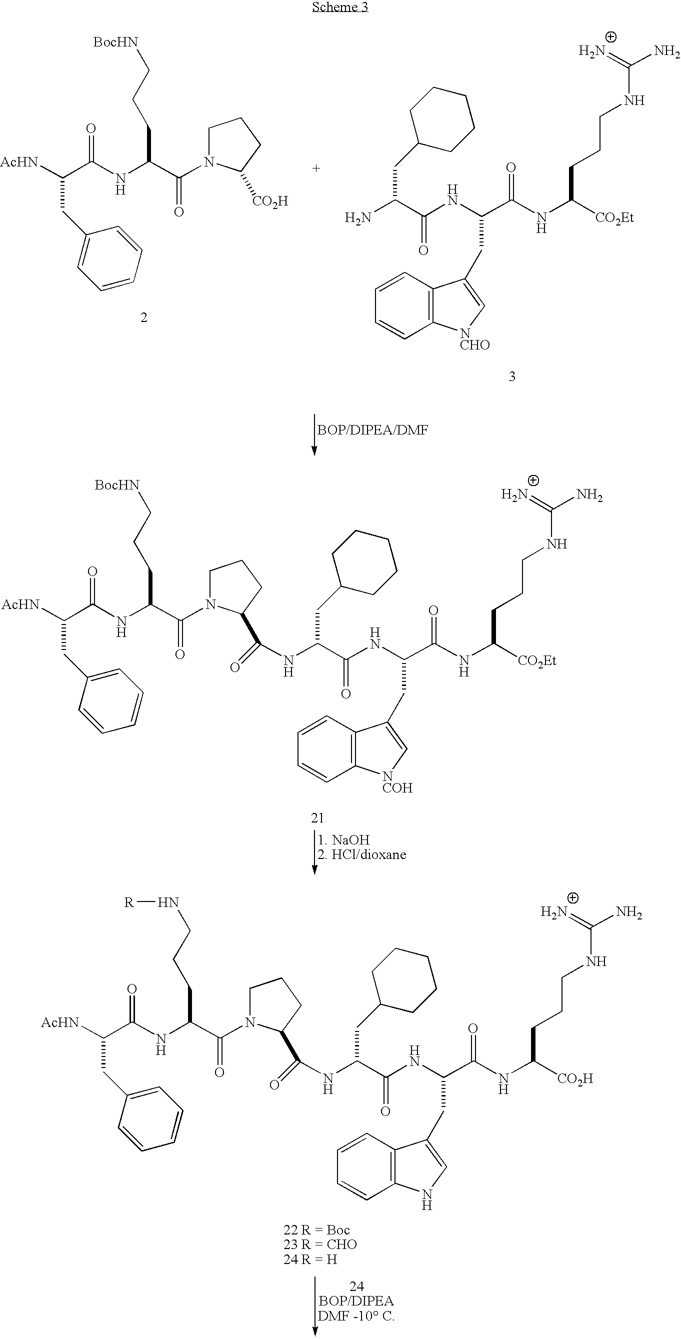

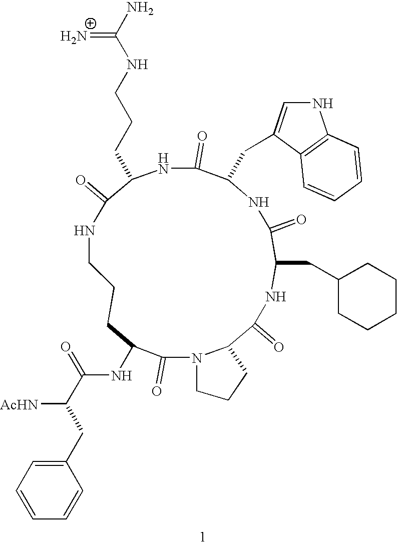

Deprotection of both the C-terminal ethyl ester and the Boc group on the ornithine side chain of 21 was required prior to the final cyclisation to 1. Hydrolysis of the C-terminal ethyl ester with concomitant loss of the formyl group from tryptophan using NaOH preferably precedes the removal of the Boc group, otherwise transfer of the formyl group from tryptophan to the ornithine side chain can occur (˜50%) giving a by-product 23 that cannot be cyclised. As the molecular weight of these isomeric formylated peptides is the same, the structure of the by-product 23 was confirmed by NMR spectroscopy where clear correlations were observed between the formyl proton and the ornithine δ-CH₂ in both the HMBC and NOESY spectra. Removal of the Boc group from the linear hexapeptide 22 was accompanied by a considerable amount (˜10%) of tert-butylation of the tryptophan residue, as a consequence of the loss of the formyl protecting group, if TFA was used even in the presence of scavengers such as water and triisopropylsilane. Efficient deprotection was achieved using a 1:1 mixture of conc. HCl/dioxane which gave only 3.6% of the tert-butylated product as shown by rpHPLC. As it was not possible to retain the protection on the indole ring, the side chains of both Trp and Arg were not protected for the final cyclisation (24 to 1, Scheme 3).

### Example 3

### Cyclisation of the Hexapeptide

The cyclisation involved the activation and coupling of a peptidyl-Arg residue (unlike a Boc-Arg residue) and was expected to proceed with some degree of racemisation. Considerable effort was directed towards minimizing the amount of racemisation, because separation of diastereomers of 1 was known to be difficult. The coupling reagents BOP and DPPA are reportedly superior to all others in terms of suppressing racemisation when cyclising or coupling peptide fragments, although there is clear evidence that some racemisation can take place^{30,31}. Table 1 summarizes cyclisation conditions used here and outcomes for the preparation of 1. Clearly the use of BOP in combination with DIPEA, especially at low temperatures, can limit racemisation to as little as 4% as determined by rpHPLC. Interestingly, the use of DBU as base caused extensive epimerisation in which the D-Arg containing macrocycle predominated by 60%, suggesting that it is a thermodynamically more stable diastereomer. DPPA caused at least 10% racemisation and the reaction was also 10 times slower than observed with BOP.

TABLE 1
Effect of Cyclisation Conditions on Racemisation

| Vial^{a} | Base^{b} | Temp.^{c} | % DE^{d} |
|---|---|---|---|
| 1 | DIPEA 4 μL 2 eq. | RT | 88.9 |
| 2 | DIPEA 10 μL 5 eq. | RT | 92.9 |
| 3 | DIPEA 4 μL 2 eq. | 0° | 93.5 |
| 4 | DIPEA 10 μL 5 eq. | 0° | 95.6 |
| 5 | DIPEA 4 μL 2 eq. | −10° C. | 95.9 |
| 6 | Pyridine 5 μL 5 eq. | RT | 81.2 (28% conversion) |
| 7 | NMM 6 μL 5 eq. | RT | 80.8 |
| 8 | NaHCO₃ 5 mg 5 eq. | RT | 88.8 |
| 9 | K₂CO₃ 8 mg 5 eq. | RT | 89.2 |
| 10 | DBU 8 μL 5 eq. | RT | 40.6 |
| 11 | No Base | RT | 81.4 (5% conversion) |
| 12 | Dimethylaniline 5 eq | RT | 82.9 (18% conversion) |
| 13 | TMEDA 2.5 eq. | RT | 89.4 |
| 14 | No Base after 24 h | RT | 82.7 (12% conversion) |

^{a}Linear hexapeptide 24 100 mg and BOP 50 mg 1 eq were dissolved in DMF 1 ml then 10 aliquots of 100 μL were taken in separate tubes.
^{b}The bases in the amounts indicated in the table were added at the ^{c}temperature also specified in the table.
^{d}After 1 hour 900 μL of 80% A:20% B was added with shaking to each tube then after brief centrifugation, 5 μL was injected into the HPLC. Linear gradient 70% A:30% B to 55% A:45% B over 30 min. Retention times: linear 24 16.2 min, cyclic 1 26.5 min, diastereomer 28.1 min.

The cyclisation of the linear hexapeptide 24 was carried out in DMF (10−1M), at low temperature (−10° C.) using DIPEA as the base and monitored by analytical rpHPLC. The reaction appeared to be strikingly clean. No trace of linear hexapeptide remained after 2 hours and the crude product, after extraction and precipitation with diethyl ether, always appeared greater than 90% pure as indicated by gradient rpHPLC (0-90% MeCN, λ214 nm). Although there were no low molecular weight by-products arising from the un-protected side chains of Arg and Trp, there was evidence for considerable amounts of polymeric material that did not elute from the rpHPLC column even with 100% MeCN. To quantify this, solutions of the analytically pure cyclic peptide 1 and the crude product were accurately prepared (5 mg/mL in 50% MeCN) and analysed by rpHPLC under the same conditions. Although both chromatograms displayed essentially only 1 peak, the integrated peak area for the crude product was only 40-60% that of the pure product, suggesting a maximum yield of only 60% w/w could be expected after purification and this indeed was found to be the case.

Previous studies have suggested that linear peptides of similar sequence to 24, and certainly the cyclic product 1, adopt turn conformations in solution that are favoured by the presence of a central proline residue and stabilized by one or more intramolecular hydrogen bonds⁵. This pre-organisation appears to greatly assist the cyclization reaction, relative to competing polymerisation, and high dilution conditions were not essential for cyclization. Comparable yields of cyclic product were obtained at concentrations of 10−1 M (49%) and 10−2 M (51%) and any benefits from further dilution were offset by the excessive solvent consumption. Purification of the final product 1 was achieved by preparative rp-HPLC after an initial adsorption step to remove polymeric material, in 33% yield and >97% purity.

### Example 4

### Boc-Orn(Z)-Pro-Ome, 6

A solution of Boc-L-Ornithine(Z)-OH (100 g, 273 mmol) in dry THF (1 L) and NMM (36 mL, 327 mmol, 1.2 equiv.) was stirred under argon and cooled to −15° C. Ethyl chloroformate (32 mL, 335 mmol, 1.2 eq) was then added while keeping the temperature at −15° C. Stirring was continued for 30 min then NMM (50 mL, 455 mmol, 1.6 eq) was added followed by a solution of H-Pro-OMe.HCl (63 g, 380 mmol, 1.4 eq) in DMF (100 mL). The mixture was allowed to warm to RT with stirring for a further 6 h. The precipitate of NMM hydrochloride was filtered off and washed with THF and the combined THF solution evaporated to dryness. The oil residue was re-dissolved in ether/DCM 3:1 (1.2 L) and washed with 2M HCl (2×300 mL), brine (300 mL), sat. NaHCO₃ (300 mL), brine (300 mL) and dried over MgSO₄. The solvent was evaporated giving the protected dipeptide as a clear, colourless oil (131 g>100%). The product contains some N-ethoxycarbonyl-Pro-OMe but this is easily removed at a later stage. This procedure gives the best yield based on the ornithine derivative which is the most expensive ingredient.

HRMS 478.2556 MH⁺ calc. for C₂₄H₃₆N₃O₇⁺ 478.2548. ¹H NMR (500 MHz, DMSO-d₆) 7.39-7.28 (m, 5H, ar), 7.24 (t, J=5.5 Hz, 1H, orn δ-NH), 6.92 (d, J=8.0 Hz, 1H, orn α-NH), 5.01 (s, 2H, OCH₂), 4.31 (dd, J=8.6, 5.0 Hz, 1H, pro α-CH), 4.15 (m, 1H, orn α-CH), 3.66 (m, 1H, pro δ-CH), 3.58 (s, 3H, OMe), 3.52 (m, 1H, pro δ-CH), 3.06-2.93 (m, 2H, orn δ-CH₂), 2.17 (m, 1H, pro β-CH), 1.90 (m, 2H, pro γ-CH₂), 1.80 (m, 1H, pro β-CH), 1.59 (m, 1H, orn β-CH), 1.64-1.40 m, 3H, orn β-CH and orn γ-CH₂), 1.36 (s, 9H, Boc). Resonances at δ 4.23 4.03, 3.93, 3.38, 1.18 and 1.09 correspond to N-ethoxycarbonyl-Pro-OMe, (separated later). Analytical rpHPLC rt=18.8 min.

### Example 5

### H-Orn(Z)-Pro-Ome, 7

The crude oily product from the above procedure (131 g containing 273 mmol of Boc-Orn(Z)-Pro-OMe) was treated with neat trifluoroacetic acid (250 mL) with swirling. CO₂ was evolved and the mixture became warm. No attempt was made to cool the mixture as it does not mix well if cold. After about 15 min the TFA was evaporated on a rotary evaporator at 40° C./20 mbar. The residue was dissolved in DCM (1.2 L), cooled to 0° C. and carefully neutralized with KOH (59 g, in water/ice 500 mL) and finally with 10% K₂CO₃ solution. The DCM layer was washed with 10% K₂CO₃ (200 mL) and the aqueous layers were back extracted with DCM (200 mL). The combined DCM layers were dried over MgSO₄ and concentrated to about 500 mL. The solution was kept cool and used immediately for the next step to avoid possible diketopiperazine formation.

HRMS 378.2026 MH⁺ calc. for C₁₉H₂₈N₃O₅⁺ 378.2024.

### Example 6

### Boc-Phe-Orn(Z)-Pro-OMe, 8

A solution of Boc-Phe-OH (75 g, 283 mmol) and NMM (32 mL, 291 mmol) in THF (1 L) was stirred under argon and cooled to −15° C. Ethyl chloroformate (26 mL, 272 mmol) was added and stirring at −15° C. was continued for 30 min. The solution prepared above containing H-Orn(Z)-Pro-OMe (273 mmol) in DCM was added and the temperature was maintained at 0° C. for 15 min then stirred at room temperature for a further 4 h. The precipitate of NMM hydrochloride was filtered off and washed with THF and the combined fractions evaporated. The oil residue was re-dissolved in ether/DCM 3:1 (1.2 L) and washed with 2M HCl (2×300 mL), brine (300 mL), sat. NaHCO₃ (300 mL), brine (300 mL) and dried over MgSO₄. The solvent was evaporated giving the protected tripeptide as a clear, colourless oil (171 g, >100%).

HRMS 625.3253 MH⁺. Calc for C₃₃H₄₅N₄O₈⁺625.3232. ¹H NMR (500 MHz, DMSO-d₆) multiple minor conformations were observed, data refers to the major conformer: 8.03 (d, J=8.0 Hz, 1H, orn α-NH), 7.39-7.12 (m, 11H, Arom and 7.27 orn δ NH), 6.89 (d, J=8.7 Hz, phe NH), 5.01 (s, 2H, OCH2), 4.53 (m, 1H, orn α-CH), 4.29 (dd, J=8.6, 5.2 Hz, 1H, pro α-CH), 4.18 (m, 1H, phe α-CH), 3.65 (m, 1H, pro δ-CH), 3.60 (s, 3H, OMe), 3.54 (m, 1H, pro δ-CH), 3.09-2.97 (m, 2H orn δ-CH), 2.94 (dd, J=13.8, 4.0 Hz, 1H, phe β-CH), 2.70 (dd, J=13.8, 10.5 Hz, 1H, phe β-CH), 2.17 (m, 1H, pro β-CH), 1.89 (m, 1H, pro β-CH), 1.82 (m, 2H pro γ-CH2), 1.69 (m, 1H, orn β-CH), 1.58-1.41 (m, 3H, orn β-CH and orn γ-CH2), 1.28 (s, 9H, Boc). Resonances at δ 4.23 4.03, 3.93, 3.38, 1.18 and 1.09 correspond to N-ethoxycarbonyl-Pro-OMe, (separated later). Analytical rpHPLC rt=20.9 min.

### Example 7

### Ac-Phe-Orn(Z)-Pro-OMe 10

The crude oily product from the above procedure (171 g, containing Boc-Phe-Orn(Z)-Pro-OMe 5 273 mmol) was treated with neat TFA (300 mL). CO₂ and heat were evolved. After a homogeneous solution had been obtained the TFA was evaporated on a rotary evaporator at 40° C./20 mbar however 120 g of TFA was retained and could not be evaporated. The residue was dissolved in DCM (1.2 L), cooled to 0° C. and carefully neutralized with KOH (59 g, in water/ice 500 mL) and finally with 10% K₂CO₃ solution. The DCM layer was washed with 10% K₂CO₃ (200 mL) and the aqueous layers were back extracted with DCM (200 mL). {free amine 9 HRMS 525.2716 MH⁺ calc for C₂₈H₃₇N₄O₆⁺ 525.2708} Acetic anhydride (27 mL, 286 mmol) was added and the solution was stirred at RT for 1 h. Mass spec. showed that complete acetylation had occurred. The solution was washed with sat. NaHCO₃ (200 mL), water (200 mL), 1M HCl (200 mL), dried over MgSO₄ and evaporated to give a viscous colourless clear oil (155 g>100%).

HRMS 567.2814 MH⁺. Calc for C₃₀H₃₉N₄O₇⁺ 567.2813. ¹H NMR (500 MHz, DMSO-d₆) 8.16 (d, J=7.7 Hz, 1H, orn-NH), 8.03 (d, J=8.4 Hz, 1H, phe-NH), 7.39-7.14 (m, 11H, Ar and Orn-δ.NH δ 7.28), 5.02 (s, 2H, OCH₂), 4.53 (m, 1H, phe α-CH), 4.48 (m, 1H, orn α-CH), 4.29 (dd, J=8.6, 5.3 Hz, pro α-CH), 3.65 (m, 1H, pro δ-CH), 3.60 (s, 3H, OMe), 3.52 (m, 1H, pro δ-CH), 3.02 (m, 2H, orn δ-CH₂), 2.96 (dd, J=13.9, 4.4 Hz, 1H, phe β-CH), 2.70 (dd, J=13.9, 10.0 Hz, 1H, phe β-CH), 2.16 (m, 1H, pro β-CH), 1.96-1.76 (m, 3H, pro γ-CH₂ and pro β-CH), 1.75 (s, 3H, Ac), 1.69 (m, 1H, orn β-CH), 1.58-1.38 m, orn γ-CH₂ and orn β-CH). Resonances at δ 4.23 4.03, 3.93, 3.38, 1.18 and 1.09 correspond to Nethoxycarbonyl-Pro-OMe, (separated later). Analytical rpHPLC rt=15.9 min.

### Example 8

### Ac-Phe-Orn(Boc)-Pro-OMe, 12

The crude oily product from the above procedure (155 g, containing Ac-Phe-Orn(Z)-Pro-OMe 7 273 mmol) was dissolved in THF (650 mL) and 2M HCl (100 mL) and hydrogenated over 10% Pd on carbon at 35 psi and room temperature for 3 h. The catalyst was filtered off and water (1 L) and ether (500 mL) were added to the filtrate and shaken. The aqueous layer was washed again with ether (300 mL) to complete the removal of neutral impurities such as Nethoxycarbonyl-Pro-OMe. The aqueous/THF solution containing Ac-Phe-Orn-Pro-OMe 11 (273 mmol) {HRMS 433.2456 calc for C₂₂H₃₃N₄O₅+433.2446} was basified with solid K₂CO₃ (30 g) then a solution of di-tert-butyl dicarbonate (60 g, 275 mmol) in THF (200 mL) was added and the solution was stirred vigorously for 1 h. The solution was extracted with ether/DCM 2:1 (3×500 mL) and the combined extracts were washed with 1M HCl (300 mL), brine (300 mL), sat. NaHCO₃ (300 mL) brine (300 mL) and dried over MgSO₄. Removal of solvent gave Ac-Phe-Orn(Boc)-Pro-OMe 9 as a colourless viscous gum 150 g>100%.

HRMS 533.2974 MH⁺ calc for C₂₇H₄₁N₄O₇⁺ 533.2970. ¹H NMR (500 MHz, DMSO-d₆) 8.14 (d, J=7.8 Hz, 1H, orn-NH), 8.02 (d, J=8.4 Hz, 1H, phe-NH), 7.28-7.15 (m, 5H, Ar), 6.80 (t, J=5.6 Hz, 1H, orn-δ.NH), 4.52 (m, 1H, phe α-CH), 4.47 (m, 1H, orn α-CH), 4.29 (dd, J=8.5, 5.0 Hz, pro α-CH), 3.65 (m, 1H, pro δ-CH), 3.61 (s, 3H, OMe), 3.54 (m, 1H, pro δ-CH), 3.00-2.84 (m, 2H, orn δ-CH₂), 2.95 (dd, J=13.9, 4.4 Hz, 1H, phe β-CH), 2.69 (dd, J=13.9, 10.0 Hz, 1H, phe β-CH), 2.16 (m, 1H, pro β-CH), 1.97-1.76 (m, 3H, pro γ-CH₂ and pro β-CH), 1.74 (s, 3H, Ac), 1.65 (m, 1H, orn β-CH), 1.54-1.36 m, orn γ-CH₂ and orn β-CH), 1.38 (s, 9H, Boc). Analytical rpHPLC rt=15.2 min.

### Example 9

### Ac-Phe-Orn(Boc)-Pro-OH, 2

The viscous gum from the above procedure (150 g containing Ac-Phe-Orn(Boc)-Pro-OMe 273 mmol) was dissolved in MeOH (500 mL) then a solution of NaOH (12 g, 300 mmol) in water (100 mL) was added. The solution was stirred at RT for 2 h and the hydrolysis was monitored periodically by mass spec. The solution was diluted with water (700 mL) and washed with ether (2×500 mL) then acidified to pH 3 with solid citric acid (60 g). The mixture was extracted with ether/DCM 2:1 (3×500 mL) then the combined extracts were washed with brine (2×300 mL) and dried over MgSO₄. Removal of solvent in vacuo gave Ac-Phe-Orn(Boc)-Pro-OH as a colourless glass (127 g, 90%). The product was crushed to a dry white powder for convenient storage. Analysis by Mass spec, NMR and HPLC showed the product to be greater than 98% pure. Microanalysis found C, 58.9; N, 10.2% C₂₆H₃₈N₄O₇ requires: C, 60.2; N, 10.8%.

HRMS 519.2847 MH⁺ calc for C₂₆H₃₉N₄O₇⁺ 519.2813. ¹H NMR (500 MHz, DMSO-d₆) 12.4 (br s, 1H, CO₂H), 8.13 (d, J=8.0 Hz, 1H, orn-NH), 8.02 (d, J=8.6 Hz, 1H, phe-NH), 7.29-7.13 (m, 5H, Ar), 6.77 (t, J=5.5 Hz, 1H, orn δ.NH), 4.52 (m, 1H, phe α-CH), 4.46 (m, 1H, orn α-CH), 4.22 (dd, J=8.7, 4.6 Hz, pro α-CH), 3.62 (m, 1H, pro δ-CH), 3.53 (m, 1H, pro δ-CH), 3.00-2.85 (m, 2H, orn δ-CH₂), 2.96 (dd, J=13.8, 4.3 Hz, 1H, phe β-CH), 2.70 (dd, J=13.8, 9.8 Hz, 1H, phe β-CH), 2.13 (m, 1H, pro β-CH), 1.95-1.78 (m, 3H, pro γ-CH₂ and pro β-CH), 1.74 (s, 3H, Ac), 1.66 (m, 1H, orn β-CH), 1.54-1.38 m, orn γ-CH₂ and orn β-CH), 1.37 (s, 9H, Boc). Analytical rpHPLC rt=13.0 min.

### Example 10

### Boc-D-Cyclohexylalanine, 15

H-D-Phenylalanine-OH (20 g, MW=165, 121 mmol) was dissolved in a 1:1 mixture of deionised water/TFA (80 mL) and to the hydrogenator vessel was added PtO₂ (800 mg, 4% w/w). The vessel was heated to 60° C. at 50 psi for 4 hrs. The solution was decanted and filtered from the catalyst and the cyclohexylalanine was precipitated out of solution by the addition of conc. HCl until no more precipitation was observed. The solid was filtered off and washed three times with acetone and air-dried. This procedure gave D-cyclohexylalanine as the HCl salt (20 g, 80% yield). The catalyst could be reused without significant reduction in reactivity for at least 5 further 20 g batches of H-D-Phenylalanine-OH before recycling. The use of TFA is preferred for quick hydrogenation times. If acetic acid is used the phenylalanine is not as soluble in the solution and was found to clog the hydrogenator lines. Total reaction time in acetic acid took two days in one experiment.

D-Cyclohexylalanine.HCl (36.5 g, 176 mmol) was dissolved in a 1:1 solution of water/THF (600 mL). Potassium carbonate (48.7 g, 352 mmol) was added and the solution was cooled to 0° C. and Boc carbonate (46 g, 1.2 eq, 211 mmol) was added over 15 min, adjusting the pH to 10-11 as the addition proceeds by adding more potassium carbonate. If the pH falls below ˜6 the unprotected amino acid precipitates out of solution as the zwitterion. When all the Boc carbonate was added, the addition of a further 100 mL of water gave a homogenous solution. This was stirred overnight at room temperature, and the THF was removed from the basic solution by rotary evaporation. The basic aqueous layer was extracted with ethyl acetate (2×100 mL) to remove unreacted Boc carbonate. The water layer was acidified to pH=2-3 by the addition of citric acid and extracted again with ethyl acetate (3×150 mL) and the combined organic layers were dried and evaporated. This procedure yields Boc-D-cyclohexylalanine (48 g, 100%) as a colourless oil which was pure by ¹H nmr and ISMS. Mass spec 272.19 MH⁺.

### Example 11

### Boc-Trp(For)-Arg-OEt, 18

Boc-Trp(For)-OH (25 g, MW=332, 75.2 mmol) was dissolved in peptide grade DMF (75 mL) and to it was added NMM (18 mL, 2 eq). The solution was cooled to −10° C. then ethyl chloroformate (7.12 mL, 75.2 mmol) added and the solution was stirred for a further 10 minutes. To this mixed anhydride was added a solution of H-Arg-OEt.2HCl (22.5 g, MW=274, 82.11 mmol) and NMM (18 mL, 2 eq) in DMF (75 mL). If H-Arg-OEt is not totally solubilised in the basic DMF solution before adding it to the mixed anhydride, then coupling may also occur at the arginine side chain giving a substantial amount of side product. The reaction was stirred for two hours allowing it to come to room temperature. This solution was subsequently poured into 10% KHSO₄ (500 mL) with vigorous stirring. A gel slowly precipitates out of solution. The solution is stirred for a further 10 minutes and the gel filtered, washed with water several times and air-dried to give the HSO₄—salt of the dipeptide (32.64 g, 70% yield). The compound was >93% pure by 1H nmr, ISMS and rpHPLC.

HRMS 517.2764 MH⁺ calc for C₂₅H₃₇N₆₀₆+517.2769; ¹H nmr δ 9.64, br. s., 1H, Arg-ηNH; 9.26, br.s., 2H, Arg-ηNH; 8.51, d, J=7.02 Hz, Arg-αNH; 8.24, s, 1H, Trp(formyl); 8.23, br.s., 1H, Trp-H7; 8.00, br.s., 1H, Trp-2H; 7.96, br. m., 1H, Arg εNH; 7.76, d, J=7.6 Hz, 1H, Trp-H4; 7.34, m, 2H, Trp-H5,H6; 7.04, d, J=8.1 Hz, 1H, Trp-αNH; 4.32, m, 1H, Trp-αH; 4.27, m, 1H, Arg-αH; 4.08, m, 2H, O—CH₂CH₃; 3.09, m, 2H, Arg-δH; 3.06, m, 1H, Trp-βH; 2.95, m, 1H, Trp-βH; 1.76, m 1H, Arg-γH; 1.68, m 1H, Arg-γH; 1.53, m, 2H, Arg βH; 1.28, s, 9H, tBu; 1.17, t, 3H, O—CH₂CH₃. Analytical rpHPLC rt=13.2 min.

### Example 12

### H-Trp(For)-Arg-OEt, 19

Boc-Trp(For)-Arg-OEt 18 was dissolved in a mixture of 90% TFA-10% water (5 mL/gram of dipeptide). The solution was stirred at room temperature for 15 minutes then the TFA was evaporated in vacuo and the product was precipitated with diethyl ether. The ether layer was decanted from the solid and the solid was washed with two further volumes of diethyl ether to remove as much of the TFA as possible. The solid was dried in vacuo and used directly for the next coupling.

### Example 13

### Boc-D-Cha-Trp(For)-Arg-OEt, 20

Boc-Trp(For)-Arg-OEt (42.00 g, 68.44 mmol) was deprotected as described above. The deprotected solid was dissolved in DMF (70 mL) and to the solution was added NMM (15 mL, 2 eq). If H-Trp(For)-Arg-OEt is not totally solubilised in the basic DMF solution before adding it to the mixed anhydride, then coupling may also occur at the arginine side-chain giving a substantial amount of side product. In a separate flask Boc-D-Cha-OH (18.18 g, 67.33 mmol) was dissolved in DMF (70 mL) and to it was added N-methylmorpholine (9 mL, 1.2 eq). The solution was cooled to −10° C. and ethyl chloroformate (6.43 mL, 67.9 mmol) was added. The reaction mixture was stirred at −10° C. for a further 15 min and to it was added the solution of HTrp(For)-Arg-OEt. The reaction mixture was stirred for a further two hours after which 10% KHSO₄ (1 L) was added. The precipitate was filtered off and washed several times with water and air-dried to give the tripeptide (40.66 g, 80% yield). The product was >90% pure by ¹H nmr, ISMS and rpHPLC.

HRMS 670.3935 MH+calc for C₃₄H₅₂N₇O₇⁺ 670.3923: ¹H nmr δ 9.65, br. s., 1H, Arg-ηNH; 9.23, br.s., 2H, Arg-ηNH; 8.47, m, 1H, Arg-αNH; 8.21, m, 1H, Trp-αNH; 8.16, br.s. 1H, Trp-H7; 8.00, br.s., 1H, Trp H2; 7.74, d, J=7.57 Hz, 1H, Trp-H4; 7.57, br. s., 1H, Arg-εNH; 7.33, m, 2H, Trp-H5,H6; 6.76, m, 1H, Cha-αNH; 4.67, br.s., 1H, Trp-αCH; 4.26, br.s., 1H, Arg-αCH; 4.07, m, 2H, O—CH₂CH₃; 3.92, m, 1H, Cha-αCH; 3.11, m, 1H, Trp-βCH: 3.09, m, 2H, Arg-δCH; 2.93, m, 1H, Trp-βCH; 1.78, m, 1H, Arg-γCH; 1.66, m, 1H, Arg-γCH; 1.55, m, 4H, Cha-δCH; 1.50, m, 2H, Arg-βCH; 1.40, m, 1H, Cha-γCH; 1.32, s, 9H, t-Bu; 1.16, t, J=7 Hz, O—CH₂CH₃; 1.12-0.91, m, 6H; Cha-βCH,εH; 0.681, m, 2H, Cha-ζCH. Mass spec 670.39 MH⁺. Analytical rpHPLC rt=17.0 min. Microanalysis found: C, 57.1; N, 13.6; S, 2.1%. Sulfate salt C₆₈H₁₀₄N₁₄O₁₈S requires: C, 56.8; N, 13.6; S, 2.2%.

### Example 14

### Ac-Phe-Orn(Boc)-Pro-D-Cha-Trp-Arg-OH, 22

Boc-D-Cha-Trp(For)-Arg-OEt. HSO₄ 21 (20.14 g, MW=766, 26.29 mmol) was deprotected according to the same procedure used for H-Trp(For)-Arg-OEt. The solid, after ether precipitation, was dissolved in DMF (50 mL) and to it was added Ac-Phe-Orn(Boc)-Pro-OH (13.07 g, 25.28 mmol) and DIPEA (4 eq, 17.2 mL) and after complete dissolution, BOP (11.18 g, 25.28-mmol). The reaction mixture was stirred overnight and to it was added 10% KHSO₄ solution (500 mL) and the aqueous layer was extracted with butan-1-ol/ethyl acetate 1:2 (3×100 mL). The combined butan-1-ol/ethyl acetate layers were extracted with 10% KHSO₄ (3×100 mL), Sat. NaHCO₃ and water (5×100 mL) and evaporated to dryness. The resultant oil was dissolved in a 1:1 mixture of water/ethanol and to it was added NaOH (2.0 g, 50 mmol) and the solution was stirred for 1 hour. The solution was poured into 10% KHSO₄ (1 L) and the resultant precipitate was extracted with butan-1-ol/ethyl acetate 1:2 (3×100 mL). The combined butanol/ethyl acetate layers were washed with water several times and the solution was evaporated in vacuo. Trituration of the oil with ether caused the compound to precipitate as a creamy solid which was filtered off and dried in an oven at 50° C. (20 g, 72%). The product was pure (>94%) by 1H nmr, ISMS and rpHPLC. Mass spec 1014 MH⁺, 507 M2H²⁺.

HRMS 1014.5765 MH⁺ calc for C₅₂H₇₆N₁₁O₁₀⁺ 1014.5771. ¹H NMR (500 MHz, DMSO-d₆) 10.76 (s, 1H, indole-NH), 8.27 (d, J=7.10 Hz, 1H, arg-NH), 8.13 (d, J=7.10 Hz, 1H, orn-NH), 8.04 (d, J=7.95 Hz, 1H, phe-NH), 8.03 (d, J=7.74 Hz, 1H, trp-NH), 7.82 (d, J=8.81 Hz, 1H, cha-NH), 7.62 (d, J=7.52 Hz, 1H, indole-H4), 7.52 (br.s., 1H, arg ε.NH), 7.30 (d, J=7.74 Hz, 1H, indole-H7), 7.27-7.14 (m, 5H, phe-Ar), 7.11 (d, J=1.93 Hz, 1H, indole-H2), 7.08 (t, J=7.52 Hz, 1H, indole-H6), 6.95 (t, J=7.52 Hz, 1H, indole-H5), 6.73 (br. s. 1H, orn ε-NH), 4.56 (m, 1H, trp α-CH), 4.52 (m, 1H, phe α-CH), 4.45 (m, 1H, orn α-CH), 4.31 (m, 1H, pro α-CH), 4.22 (m, 1H, arg α-CH), 3.56 (m, 2H, pro δ-CH₂), 3.19-3.06 (m, 3H, arg δ-CH2, trp β-CH), 2.98-2.91 (m, 2H, phe β-CH, trp β-CH), 2.89 (m, 2H, orn δ-CH₂), 2.70 (dd, 1H, J=9.67, 13.75 Hz, phe β-CH), 1.98 (m, 1H, pro β-CH), 1.84 (m, 1H, pro γ-CH), 1.80-1.71 (m, 2H, pro γ-CH, arg γ-CH), 1.75 (s, 3H, acetyl-CH₃), 1.70-1.63 (m, 3H, arg γ-CH, orn γ-CH, pro β-H), 1.54 (m, 2H, arg β-CH₂), 1.5-1.38 (m, 7H, orn γ-CH, orn β-CH2, c-hexyl 4H) 1.36 (s, 9H, t-Butyl), 1.35 (m, 1H, c-hexyl), 1.15 (t, 2H, cha β-CH₂), 1.07-0.93 (m, 4H, c-hexyl), 0.70 (m, 2H, c-hexyl). Analytical rpHPLC rt=15.8 min.

The fully protected hexapeptide before de-esterification was hygroscopic and difficult to handle. It was found that the partially deprotected product dries to a non-hygroscopic powder. The reaction was repeated several times giving product in 80% yield.

### Example 15

### Ac-Phe-Orn-Pro-Cha-Trp-Arg-OH, 24

Ac-Phe-Orn(Boc)-Pro-D-Cha-Trp-Arg-OH (100 g, 90 mmol) was added to a solution of conc. HCl/dioxane (200 mL) and stirred at room temperature for 1 hour. The solution was cooled to 0° C. and to it was slowly added a 4M NaOH solution (˜70 mL) until the pH of the reaction mixture was ˜7. The resultant neutral solution was extracted with butanol/ethyl acetate 1:2 (3×300 ml) and the combined layers were washed with water (2×50 mL). Evaporation of the solvent and trituration with ether gave the fully unprotected linear peptide as an off-white powder (91 g, 95%). The product is greater than 93% pure by 1H nmr, ISMS and rpHPLC. Mass spec 914.53 MH⁺ 457.77 M2H²⁺.

HRMS 914.5269 MH⁺ calc for C₄₇H₆₈N₁₁O₈⁺ 914.5247. ¹H NMR (500 MHz, DMSO-d₆) 10.77 (s, 1H, Indole-NH), 8.36 (d, J=7.6 Hz, 1H, arg-NH), 8.26 (d, J=8.06 Hz, 1H, orn-NH), 8.04 (d, J=8.17 Hz, 1H, phe-NH), 8.01 (d, J=8.28 Hz, 1H, trp-NH), 7.90 (d, J=8.50 Hz, 1H, cha-NH), 7.66 (br.s., 2H, orn-NH2), 7.63 (d, J=8.07 Hz, 1H, indole-H4), 7.54 (t, J=5.56 Hz, 1H, arg ε.NH), 7.31 (d, J=7.96 Hz, 1H, indole-H7), 7.27-7.15 (m, 5H, phe-Ar), 7.13 (d, J=1.85 Hz, 1H, indole-H2), 7.04 (t, J=7.30 Hz, 1H, indole-H6), 6.96 (t, J=7.52 Hz, 1H, indole-H5), 4.57 (m, 1H, trp α-CH), 4.50 (m, 2H, phe α-CH & orn α-CH), 4.30 (m, 2H, pro α-CH & cha α-CH), 4.22 (m, 1H, arg α-CH), 3.54 (m, 2H, pro b-CH₂), 3.18-3.07 (m, 3H, arg δ-CH₂, trp β-CH), 2.96-2.87 (m, 2H, phe β-CH, trp β-CH), 2.77-2.67 (m, 3H, orn δ-CH₂, phe β-CH), 2.02 (m, 1H, pro β-CH), 1.90-1.62 (m, 7H, pro β-CH, pro γ-CH₂, arg β-CH₂, orn γ-CH₂), 1.75 (s, 3H, acetyl-CH3), 1.61-1.50 (m, 8H, arg γ-CH₂, orn β-CH₂, c-hexyl 4H), 1.39 (d, 1H, c-hexyl), 1.15 (t, 2H, cha β-CH₂), 1.08-0.93 (m, 4H, c-hexyl), 0.70 (m, 2H, c-hexyl). Analytical rpHPLC rt=11.7 min, Peak at rt=13.5 is tert-butylated material.

### Example 16

### Ac-Phe-[Orn-Pro-D-Cha-Trp-Arg], 1 (TFA Salt)

A solution of the fully deprotected hexapeptide Ac-Phe-Orn-Pro-Cha-Trp-Arg-OH (100 g, 105 mmol) in DMF (1 L) and diisopropylethylamine (100 mL, 570 mmol, 5.5 eq) was stirred at room temperature until homogeneous then cooled to −10° C. BOP (solid 50 g, 113 mmol, 1.08 eq) was added and the solution was stirred at −10 to −5° C. for 2 h. The DMF was evaporated and the residue was dissolved in 1-butanol/EtOAc 3:1 (1 L) and washed with brine (300 mL), 2M HCl (300 mL) and water (2×300 mL). The solvent was evaporated in vacuo and the residue was triturated with ether giving a pale cream solid 98 g. The solid product was filtered off and washed on the filter with ether and dried under high vacuum. The crude product dries to a non-hygroscopic powder. Analysis of the crude cyclic peptide by analytical HPLC shows the desired product together with a minor diastereomer in the ratio 96:4. The cyclisation has been done on 2 batches of 100 g with similar results. The crude product was dissolved in 50% MeCN/50% water (1 L) and TFA (10 mL) with stirring and warming at approximately 40° C. for 15 min. The cloudy solution was applied to a column of reverse phase C18 silica gel (Fluka cat. 60757) of depth 10 cm contained in a sintered glass filter funnel of diameter 10 cm and light vacuum was applied. The column was eluted with a further 500 mL of 50% MeCN/50% water then the combined eluent was partially evaporated on a rotary evaporator until the product began to precipitate. A small volume of MeCN was added sufficient to redissolve the precipitate then the solution was applied in 50 mL aliquots to a preparative HPLC column (Vydac C18 300 Å, 50×250 mm) and eluted with 38% MeCN/61.9% water/0.1% TFA at 70 mL/min with UV detection at 280 nm. The peak containing the cyclic peptide (retention time 18-23 min) was fractionated into 6 separate vessels that were analysed for diastereomer content by analytical HPLC (Analytical HPLC conditions: Column: Vydac peptide & protein 300 Å 5 μM 4.6×250 mm Flow rate 1 mL/min. 70% A:30% B to 55% A:45% B over 30 min. where buffer A is water+0.1% TFA, buffer B is 90% MeCN/10% water+0.1% TFA. Retention times: linear peptide 16.2 min, cyclic product 1 26.5 min, minor diastereomer 28.1 min). Pure fractions were combined and lyophilised giving a white powder 33 g. Mass spec 896.52 MH⁺, 448.76 M2H²⁺.

HRMS 896.5105 MH⁺ calc for C₂₆H₃₉N₄O₇⁺ 896.5141. Microanalysis found C, 56.8; N, 15.5%. TFA salt C₄₉H₆₆F₃N₁₁O₉ requires C, 58.3; N, 15.3%. Analytical rpHPLC rt=14.3 min.

TABLE 2
¹H NMR (500 MHz, DMSO-d₆) for 1

| Residue | NH | [symbol] J_{NH—CH}_{₃} | Hα | Hβ | Hγ | Others |
|---|---|---|---|---|---|---|
| AcPhe | 8.11 | 8.4 | 4.50 | 2.69.2.96 | | 1.74(Me).7.23 |
| Orn | 7.96 | 7.0 | 4.55 | 1.44.1.64 | 1.22 | 2.76.3.38.7.04 |
| | | | | | | (NH [symbol] ) |
| Pro | — | — | 4.57 | 1.63.1.99 | 1.63 | 3.41.3.62 |
| dCha | 8.17 | 4.6 | 4.01 | 1.15.1.26 | 0.93 | 0.62.0.69 |
| Trp | 8.41 | 7.0 | 4.23 | 2.96.3.26 | | 7.15.7.39.10.86 |
| | | | | | | (NH) |
| Arg | 7.82 | 8.2 | 4.12 | 1.63.1.87 | 1.50 | 3.11.7.59 |
| | | | | | | (NH [symbol] ) |

### Example 17

### Ac-Phe-[Orn-Pro-D-Cha-Trp-Arg], 1 (Acetate Salt)

The crude cyclised product was dissolved in 50% MeCN/50% water (1 L) and glacial acetic acid (10 mL) and applied to a 10×10 cm precolumn of reverse phase C18 silica gel as described above for the TFA salt. The solution obtained was purified by preparative HPLC using a solvent consisting of 38% MeCN/62% water, sodium acetate 6 g/L, adjusted to pH 6 with glacial acetic acid at 70 mL/min. Fractions containing the pure cyclic peptide were combined and partially evaporated to remove as much MeCN as possible without causing precipitation of the product. The solution (500 mL aliquots) was applied to a preparative HPLC column (Vydac C18 300 Å, 50×250 mm) previously equilibrated with 1% AcOH in water and eluted with 1% AcOH in water at 70 mL/min for 30 min to complete the de-salting process. The product was quickly removed from the column by elution with 50% MeCN/49% water/1% AcOH and the solution was lyophilised giving the acetate salt as a white powder 31 g. Mass spec 896.52 MH⁺, 448.76 M2H²⁺. Analytical rpHPLC rt=14.3 min.

### Example 18

### Purification

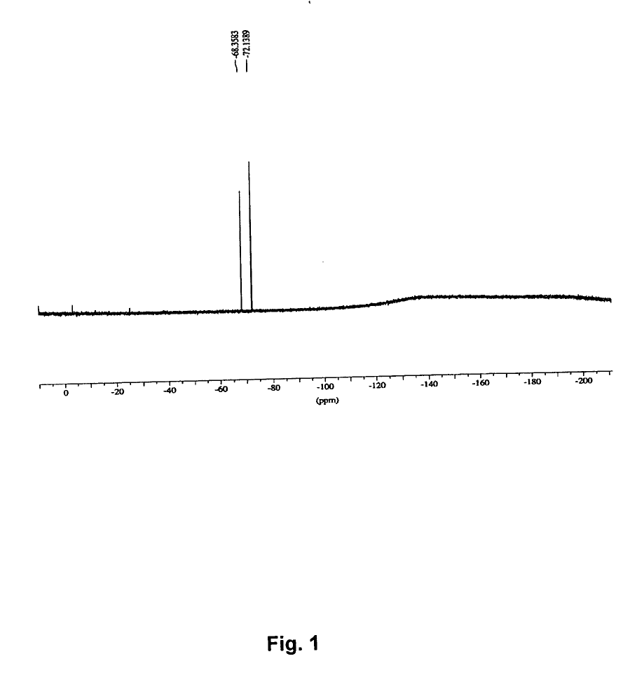

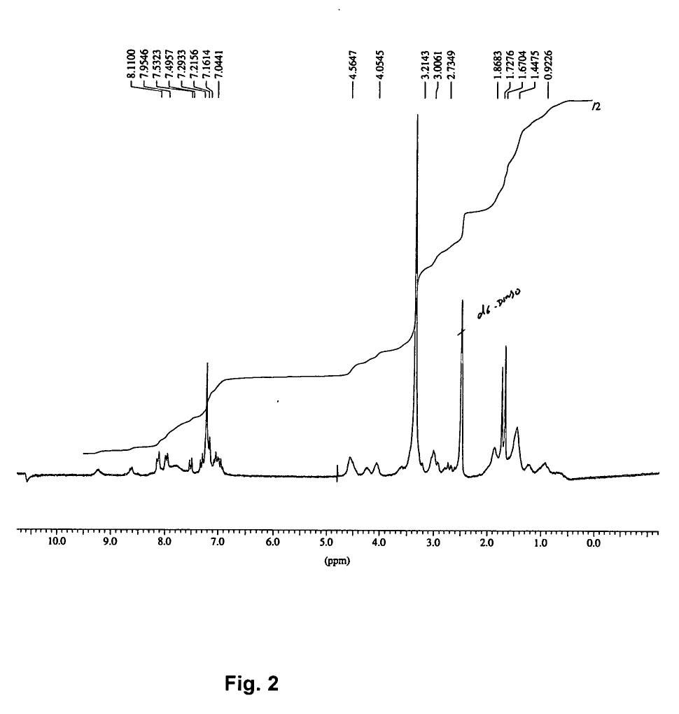

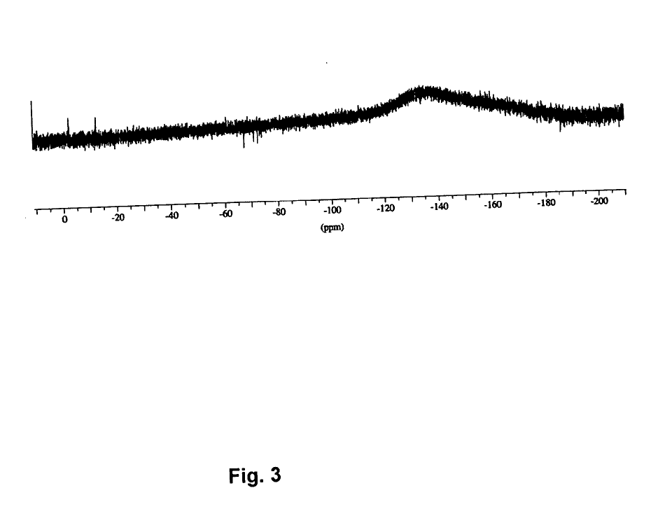

The crude product of Example 15 was purified by prep HPLC using 0.5% AcOH in water and 0.5% AcOH in 90% ACN in water as buffers. The crude product was purified in a gradient program and major fraction was collected. After lyophilization, it gave a white powder which failed the solubility test at 10 mg/ml. ¹⁹F NMR confirmed that there were still two TFA salts in the formula (FIG. 1). This product was repurified on the prepHPLC once more, the major fraction was collected and lyophilized to give 3D53. 12 mg of this product dissolved in 1 ml water gave a clear solution. This product was analysed by ¹H and ¹⁹F NMR. ¹H NMR gave two singlet at 1.73 and 1.73 (FIG. 2) and ¹⁹F NMR showed there was no TFA salt in the product (FIG. 3).

Conclusions

Examples 1 to 17 describe processes that can be utilised for the medium-large scale synthesis of cyclic peptides, without purification of intermediates. The convergent synthesis described above shows that arginine side-chain protection is not required and that in this case, the simple precipitation of intermediates provides products of sufficient purity to carry out subsequent reactions efficiently. The tripeptides were coupled at the Pro-Cha junction to minimize racemization via the oxazolone pathway. Adequate quantities of the final product (64 g) could be purified by rp-HPLC to >97% purity in an overall yield of 12.5% from the commercially available amino acids.

### Example 19

### Larger Scale Preparation of PMX53

A new method has also been developed for the larger scale production of PMX53 in solution phase synthesis using HBTU as the major coupling reagent and DIPEA as the base.

Since both ZOrn(Boc)OH and AcPheOH are commercially available, they were chosen as the starting materials instead of BocOrn(Z)OH and BocPheOH which eliminated two steps from the synthesis. The coupling reaction to prepare the intermediate of dipeptides and tripeptide was successful using HBTU/DIPEA without Ar or N₂ protection (see Schemes 4 and 5). Purification was not conducted for any of the intermediates. The deprotection conditions for removal of the Boc group were modified using TFA in DCM or thioanisole. A method has been developed to convert PMX53.2TFA salt to PMX53.2HOAc salt in a more effective way by using Amberlite IRA 410 ion exchange resin, with 10% AcOH (aq) used as an elution. The different salt forms of PMX53 have dramatically different solubilities which can effect its biological activity in animal models of disease. This preparation method of PMX53.2HOAc will have great benefits for manufacturing the compound on a larger scale, the overall yield is 25%. 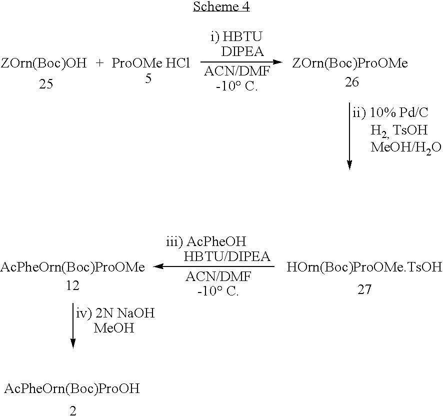 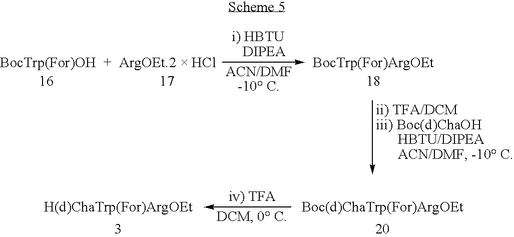
ZOrn(Boc)ProOMe

ZOrn(Boc)OH was dissolved in ACN at −5° C., then DIPEA was added, followed by HBTU which was dissolved in DMF/CAN. The resulting solution was added to the solution of HCl.ProOMe that was neutralized by DIPEA (1 eq.) at −5±2° C. in ACN. Extra DIPEA was added to keep the pH at 7.8. The reaction was completed within an hour. The solvent was evaporated under reduced pressure and the residue was dissolved in EtOAc, then washed with 10% K₂CO₃, 10% KHSO₄ and brine and dried with MgSO₄. Evaporating the solvent gave the crude product, ZOrn(Boc)ProOMe, as a colorless oil in quantitative yield. HPLC showed the crude product was quite pure and no further purification was needed at this stage. HRMS give 500.2374 as [M+Na]⁺. 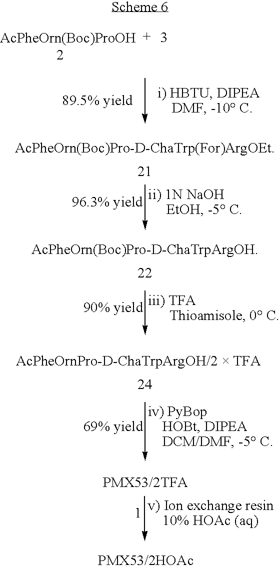
HOrn(Boc)ProOMe

ZOrn(Boc)ProOMe was dissolved in MeOH/H₂O. TsOH (1 eq.) and 10% Pd/C (5% of SM) were added to the solution. After degassing, the solution was saturated with hydrogen at room temperature for 2 hrs. Additional 10% Pd/C was needed to achieve 100% conversion and stirred for 2 more hours. When the reaction was complete, another 0.05 eq. of TsOH was added to keep the pH<4, the Pd/C was filtered through a celite cake and the filtrate was concentrated to give the crude product, HOrn(Boc)ProOMe.TsOH, as white solid in quantitative yield. HPLC showed the purity was greater than 96%.

AcPheOrn(Boc)ProOMe

To a solution of AcPheOH in ACN at −10° C. was added DIPEA (1 eq.), followed by HBTU (1 eq.) in DMF. HOrn(Boc)ProMe (0.8 eq) in ACN was added to the solution, then DIPEA (0.8 eq) was added. Extra DIPEA was added to keep the pH>7.4. The reaction was completed in an hour. After removal of ACN under reduced pressure, the residue was dissolved in EtOAc. The organic layer was washed with 5% NaHCO₃/8% NaCl, and then dried with MgSO₄ yielding the crude product, AcPheOrn(Boc)ProOMe, as a light yellow solid. HRMS gave 555.2792 as [M+Na]⁺.

AcPheOrn(Boc)ProOH

Saponification of AcPheOrn(Boc)ProOMe with 3N NaOH (1.2 eq.) in MeOH was performed at 0° C.—rt O/N. After neutralization with 2N HCl, methanol was evaporated under reduced pressure. The residue was dissolved in DCM and the mixture was acidified to pH 2.5 with 2N HCl. The aqueous phase was extracted with DCM twice more. The combined DCM extracts were washed with 10% NaCl three times and dried with MgSO₄. Evaporation of the solvent yielded the crude product, AcPheOrn(Boc)ProOH, as a light yellow solid. HRMS gave 541.2642 as [M+Na]⁺, cal for C₂₆H₃₈N₄O₇Na⁺ 541.2638.

BocTrp (For) ArgOEt

To a suspension of 2HCl.ArgOEt in ACN/DMF was added DIPEA (1 eq). BocTrpOH was suspended in CAN at 0° C., then DIPEA (1 eq) was added, following by HBTU in DMF. This solution was added to the ArgOEt suspension. Extra DIPEA was needed to keep the pH<6.5. The reaction was complete in 1.5 hrs. ACN was evaporated, then the residue was poured slowly into 10% KHSO₄ and stirred for an hour. The precipitate was filtered and washed several times with brine and water and dried in the air. However, because of the length of time required for the product to dry, it was found to be quicker to dissolve of the crude product in methanol, then evaporate the solvent and repeat the process one more time. After drying in vacuo, it gave the crude product, BocTrp(For)ArgOEt.HSO₄ as a white solid with greater 95% purity and more than 73% yield. HRMS gave 517.2772 as [M+1]⁺, cal for C₂₅H₃₇N₆O₆⁺ 517.2769.

HTrp(For)ArgOEt

To a suspension of BocTrp(For)ArgOEt in DCM at 0° C. was added TFA (23 eq) dropwise. After stirring for 3 hrs, HPLC showed the reaction was completed. The reaction mixture was poured into cold ether and the precipitate was filtered and washed with more ether. Drying in vacuo, gave the crude product, HTrp(For)ArgOEt.2TFA in 83% yield. HRMS gave 417.2251 as [M+1]⁺. Cal for C₂₀H₂₈N₆O₄ 417.2250.

Boc-D-ChaTrp(For)ArgOEt

To a suspension of HTrp(For)ArgOEte2TFA in DMF was added DIPEA (2 eq) at −5° C. Boc(d)ChaOH (DCHA) was dissolved in DMF at 0° C., then HBTU was added protionwise, The resulting mixture was added to the solution of HTrp(For)ArgOEt. Extra DIPEA was added to keep the pH>7.0. The reaction was complete in an hour. The reaction mixture was poured into 1.5% KHSO₄ (aq) and stirred for 30 min. The precipitate was filtered and washed several times with water until the pH was close to 6, then washed twice with Et₂O. Drying under vacuo, gave the crude product with a yield of 98%. HPLC showed the purity was greater than 98%. HRMS gave 670.3934 as [M+1]⁺, cal for C₃₄H₅₂N₇O₇ 670.3923.

H-D-ChaTrp(For)ArgOEt

This product was obtained using the same deprotection procedure as used for H(d)ChaTrp(For)ArgOEt and resulted in 87% yield. ES/MS gave 417 as [M+1]⁺.

AcPheOrn(Boc)Pro(d)ChaTrp(For)ArgOEt

HBTU was applied for the coupling reaction which give the product in 90% yield. HRMS gave 1070.6060 as [M+1]⁺ cal for C₅₅H₈₀N₁₁O₁₁ 1070.6038.

AcPheOrn(Boc)Pro-D-ChaTrpArgOH

Saponification of

AcPheOrn(Boc)Pro(d)ChaTrp(For)ArgOEt with 1N NaOH in EtOH/Et₂O gave the title compound in 96% yield. HRMS gave 1036.5613 as [M+Na]⁺. Cal for C₅₂H₇₅N₁₁O₁₀ Na 1036.5596.

AcPheOrnPro-D-ChaTrpArgOH

To the solid of AcPheOrn(Boc)Pro-D-ChaTrpArgOH was added a mixture of TFA (3 ml/g of SM)/thioanisole (0.5 ml/g of SM) at 6±2° C. The deprotection was completed in an hour and the reaction mixture was then poured into cold diethyl ether; the precipitate was filtered and washed with additional diethyl ether and dried in vacuo. It gave the crude product, AcPheOrnPro(d)ChaTrpArgOH, with a purity >95%, which was ready for the next step of cyclisation without further purification.

AcPhe[OrnPro-D-ChaTrpArg].2TFA (PMX53/.2TFA)

Cyclisation of AcPheOrnPro-D-ChaTrpArgOH was performed at −5° C. in DMF using PyBOP and HOBt as the activation and coupling reagent and DIPEA as the base. The reaction was completed in three hours. The reaction mixture was poured into the cold Et₂O. The precipitate was filtered and washed with more Et₂O. After drying, it gave the crude product in about 74% yield.

AcPheOrnPro-D-ChaTrpArgOH.2HOAc (PMX53.2HOAc)

The crude product PMX53.2TFA was firstly converted to the PMX53.2HOAc salt by using Amberlite IRA 410 ion exchange resin, with 10% AcOH in water was used as an elution. The combined fractions were lyophilized to give a crude product that was further purified by prepHPLC. This method can be applied to other cyclic peptide TFA salts which are obtained from solid phase peptide synthesis, but it has to be performed twice to convert to its HOAc salt.

Other coupling reagents a such as EDC, DCC and TOTU were also tested to form dipeptide or tripetide.

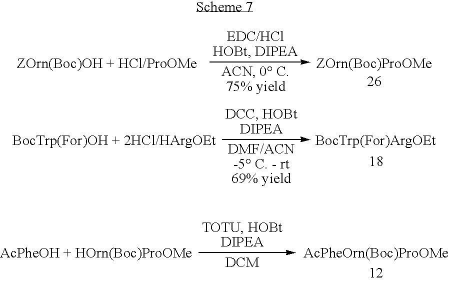

While the invention has been described in the examples with respect to the preparation of cyclic peptide 1, it will be appreciated that the same process could be used to prepare other cyclic peptides of formula I.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

The following references, to the extent that they provide exemplary procedural or other details supplementary to those set forth herein, are specifically incorporated herein by reference.
1. Makrides, S. C. Pharmacol Rev 1998, 50, 59-87.
2. Taylor, S. M.; Fairlie, D. P. Exp. Opin. Ther. Patents 2000, 10, 449-458.
3. Webpage for AVANT Immunotherapeutics, Inc. Complement Inhibitors, http://www.avantimmune.com/technology.htm
4. Wong, A. K.; Finch, A. M.; Pierens, G. K.; Craik, D. J.; Taylor, S. M.; Fairlie, D. P. J Med Chem 1998, 41, 3417-25.
5. Finch, A. M.; Wong, A. K.; Paczkowski, N.J.; Wadi, S. K.; Craik, D. J.; Fairlie, D. P.; Taylor, S. M. J Med Chem 1999, 42, 1965-74.
6. Paczkowski, N.J.; Finch, A. M.; Whitmore, J. B.; Short, A. J.; Wong, A. K.; Monk, P. N.; Cain, S. A.; Fairlie, D. P.; Taylor, S. M. Br J Pharmacol 1999, 128, 1461-6.
7. Woodruff, T. M.; Strachan, A. J.; Sanderson, S. D.; Monk, P. N.; Wong, A. K.; Fairlie, D. P.; Taylor, S. M. Inflammation 2001, 25, 171-7.
8. Haynes, D. R.; Harkin, D. G.; Bignold, L. P.; Hutchens, M. J.; Taylor, S. M.; Fairlie, D. P. Biochem Pharmacol 2000, 60, 729-33.
9. Cain, S. A.; Woodruff, T. M.; Taylor, S. M.; Fairlie, D. P.; Sanderson, S. D.; Monk, P. N. Biochem Pharmacol 2001, 61, 1571-9.
10. Short, A.; Wong, A. K.; Finch, A. M.; Haaima, G.; Shiels, I. A.; Fairlie, D. P.; Taylor, S. M. Br J Pharmacol 1999, 126, 551-4.
11. Woodruff, T. M.; Strachan, A. J.; Dryburgh, N.; Shiels, I. A.; Reid, R. C.; Fairlie, D. P.; Taylor, S. M. Arthritis Rheum 2002, 46, 2476-85.
12. Strachan, A. J.; Shiels, I. A.; Reid, R. C.; Fairlie, D. P.; Taylor, S. M. Br J Pharmacol 2001, 134, 1778-86.
13. Strachan, A. J.; Woodruff, T. M.; Haaima, G.; Fairlie, D. P.; Taylor, S. M. J Immunol 2000, 164, 6560-5.
14. Arumugam, T. V.; Shiels, I. A.; Strachan, A. J.; Abbenante, G.; Fairlie, D. P.; Taylor, S. M. Kidney Int 2003, 63, 134-42.
15. Arumugam, T. V.; Shiels, I. A.; Woodruff, T. M.; Reid, R. C.; Fairlie, D. P.; Taylor, S. M. J Surg Res 2002, 103, 260-7.
16. Bernardelli, P.; Gaudilliere, B.; Vergne, F. Annual Reports in Medicinal Chemistry, Vol 37 2002, 37, 257-277.
17. Gaudilliere, B.; Berna, P. Annual Reports in Medicinal Chemistry, Vol 35 2000, 35, 331-356.
18. Gaudilliere, B.; Bernardelli, P.; Berna, P. Annual Reports in Medicinal Chemistry, Vol 36 2001, 36, 293-318.
19. Galatsis, P. Annual Reports in Medicinal Chemistry, Vol 32 1997, 32, 305-326.
20. Cheng, X. M. Annual Reports in Medicinal Chemistry, Vol 31 1996, 31, 337-355.
21. Stewart, P. M. Trends in Endocrinology and Metabolism 2000, 11, 128-132.
22. Brady, S. F.; Freidinger, R. M.; Paleveda, W. J.; Colton, C. D.; Homnick, C. F.; Whitter, W. L.; Curley, P.; Nutt, R. F.; Veber, D. F. Journal of Organic Chemistry 1987, 52, 764-769.
23. Zhang, L. H.; Pesti, J. A.; Costello, T. D.; Sheeran, P. J.; Uyeda, R.; Ma, P.; Kauffman, G. S.; Ward, R.; McMillan, J. L. Journal of Organic Chemistry 1996, 61, 5180-5185.
24. Chan, W. C.; White, P. D. Fmoc solid phase peptide synthesis: a practical approach; Oxford University Press: New York, 2000.
25. Fields, G. B.; Noble, R. L. Int J Pept Protein Res 1990, 35, 161-214.
26. Jones, J. The chemical synthesis of peptides; Clarendon Press; New York: Oxford University Press: Oxford, 1991.
27. Schuda, P. F.; Greenlee, W. J.; Chakravarty, P. K.; Eskola, P. Journal of Organic Chemistry 1988, 53, 873-875.
28. Beames, D. J.; Mander, L. N. Aust. J. Chem. 1974, 27, 1257-68.
29. Bodanszky, M.; Bodanszky, A. The practice of peptide synthesis; 2nd, rev. ed.; Springer-Verlag: Berlin; New York, 1994.
30. Benoiton, N. L.; Lee, Y. C.; Steinaur, R.; Chen, F. M. Int J Pept Protein Res 1992, 40, 559-66.
31. Steinauer, R.; Chen, F. M.; Benoiton, N. L. Int J Pept Protein Res 1989, 34, 295-8.

1. A process for the preparation of a compound of formula I 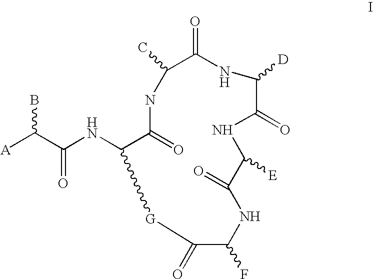
in which
A is H, NH₂, optionally substituted alkyl, optionally substituted aryl, NH acyl, NH optionally substituted alkyl, N(optionally substituted alkyl)₂ or NH succinate;
B is optionally substituted alkyl or optionally substituted aryl;
C is an optionally protected amino acid side chain;
D is an optionally protected amino acid side chain;
E is an optionally protected amino acid side chain; optionally substituted aryl; or optionally substituted heteroaryl;
F is an optionally protected D- or L-amino acid side chain selected from the group consisting of arginine, homoarginine, citrulline, homocitrulline, glutamine, lysine and canavanine; and
G is an optionally protected D- or L-amino acid side chain selected from the group consisting of ornithine and lysine,
or pharmaceutically acceptable salts, derivatives, hydrates, solvates, prodrugs, tautomers and/or isomers thereof
which process comprises the steps of:
(a) coupling an optionally protected compound of formula II 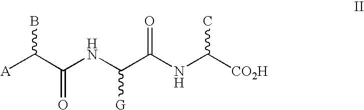
in which A, B, C and G are as defined in formula I with an optionally protected compound of formula III 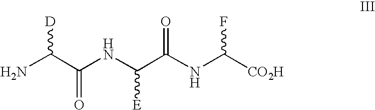
in which D, E and F are as defined in formula I to form an optionally protected compound of formula IV 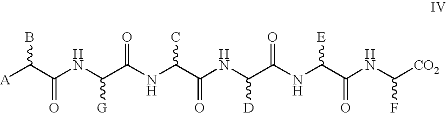
in which A, B, C, D, E, F and G are as defined in formula I; and
(b) cyclising the compound of formula IV.

2. A process according to claim 1, in which A is NH acyl or NH succinate.

3. A process according to claim 1, in which the optionally substituted aryl in B is an optionally substituted phenyl or an optionally substituted benzyl.

4. A process according to claim 1, in which the optionally substituted aryl in B is phenyl, benzyl, 4-nitrophenyl, 4-aminophenyl, 4-dimethylaminophenyl, halophenyl or phenyl-(CH₂)ₙ in which n is an integer from 2 to 5.

5. A process according to claim 1, in which C is an optionally protected side chain of L- or D-amino proline or hydroxyproline.

6. A process according to claim 1, in which D is an optionally protected side chain of L- or D-cyclohexane amino acid.

7. A process according to claim 1, in which E is an optionally protected side chain of L- or D-tryptophan or alanine.

8. A process according to claim 1, in which the optionally substituted aryl in E is an optionally substituted naphthyl or an optionally substituted benzothienyl.

9. A process according to claim 1, in which the compound of formula I is Ac-Phe[Orn-Pro-D-Cha-Trp-Arg] (3D53).

10. A process according to claim 1, in which step (a) and/or step (b) involve the use of a coupling agent and a base.

11. A process according to claim 10, in which the coupling agent is benzotriazol-1-yloxy-tris(dimethylamino)-phosphonium hexafluorophosphate (BOP).

12. A process according to claim 10, in which the base is diphenylphosphonyl azide (DPPA) for step (a) and selected from DPPA, diisopropylethylenediamine (DIPEA), NaHCO₃ and tetramethylethylenediamine (TMEDA) for step (b).

13. A process according to claim 10, in which step (b) is performed at temperatures of about −10° C. to about room temperature.

14. A process according to claim 10, in which the compound of formula I is purified using preparative HPLC.

15. A process according to claim 10, in which the compounds of the formulae II, III and IV are AcPhe-Orn(Boc)-Pro-OH 2, D-Cha-Trp(For)-Arg-OEt 3 and compounds 22-24 shown below, respectively.

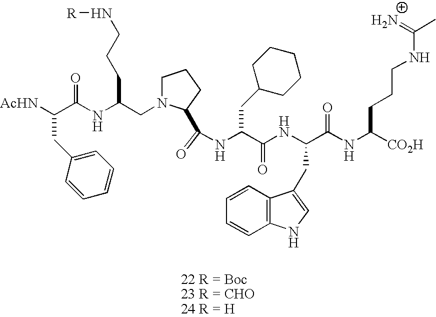

16. A compound of formula I prepared by the process as defined in claim 1.

17. Compounds of formulae II and III as defined in claim 1.

18. A process for the preparation of the compound of formula II, as defined in claim 1, which comprises coupling an optionally protected compound of the formula V, 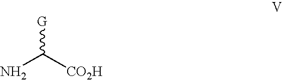
in which G is as defined in formula I according to claim 1,
and an optionally protected compound of the formula VI, 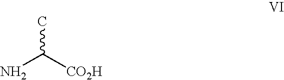
in which C is as defined in formula I,
and an optionally protected compound of the formula VII 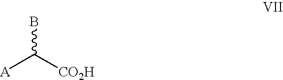
in which A and B are as defined in formula I.

19. A process according to claim 18, in which the compound of formula V is first coupled with the compound of formula VI to form a dipeptide which is then coupled to the compound of formula VII.

20. A process for the preparation of the compound of formula III, as defined in claim 1, which comprises coupling an optionally protected compound of formula VIII, 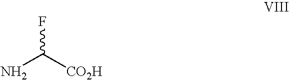
in which F is as defined in formula I according to claim 1,
and an optionally protected compound of formula IX, 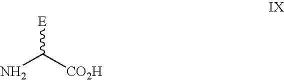
in which E is as defined in formula I,
and an optionally protected compound of formula X, 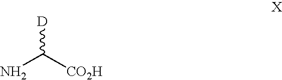
in which D is as defined in formula I.

21. A process according to claim 20, in which the compound of formula VIII is first coupled with the compound of formula IX to form a dipeptide which is then coupled to the compound of formula X.

22. A process according to claim 18 or claim 20, in which the coupling step is performed using a coupling agent and a base.

23. A process according to claim 22, in which the coupling agent is ethyl chloroformate, N,N,N′,N′-tetramethyl-O-(1H-benzotriazol-1-yl)uranium hexafluorophosphate (HBTU), O[ethoxycarbonyl) cyanomethylenamino]N,N,N′,N′-tetramethyl uranium tetrafluoroborate (TOTU), N-(3-dimethylaminopropyl)-N′-ethylcarbodiimide hydrochloride (EDC) or N,N′-dicyclohexycarbodiimide (DCC).

24. A process according to claim 23, in which the coupling agent is HBTU.

25. A process according to claim 22, in which the base is N-methyl morpholine (NMM) or DIPEA.

26. A process according to claim 25, in which the base is DIPEA.

27. A process according to claim 18, in which the compounds of the formulae V, VI and VII are Boc-Orn(Cbz)-OH 4, H-Pro-OMe 5 and Boc-Phe-OH 13, respectively.

28. A process according to claim 20, in which the compounds of the formulae VIII, IX and X are H-Arg-OEt.2HCl 17, Trp(For)-OH 16 and Boc-D-cyclohexylalanine 15, respectively.
